(19) United States
(12) Patent Application Publication
(10) Pub. No.: US 2017/0242036 A1
(43) Pub. Date: Aug. 24, 2017

(54) PROCESSES AND KITS TO DETECT AND MONITOR FOR DIAGNOSTIC BIOMARKERS FOR POST TRAUMATIC STRESS DISORDER (PTSD) AND TO DIFFERENTIATE BETWEEN SUICIDAL AND NON-SUICIDAL FORM OF THE DISORDER

(71) Applicants: BANYAN BIOMARKERS, INC., Alachua, FL (US); THE HENRY M. JACKSON FOUNDATION FOR THE ADVANCEMENT OF MILITARY MEDICINE, INC., Bethesda, MD (US)

(72) Inventors: Harvey POLLARD, Potomac, MD (US); Lei ZHANG, Germantown, MD (US); Ofer EIDELMAN, Bethesda, MD (US); Robert J. URSANO, Kensington, MD (US); He LI, Rockville, MD (US); Tung-Pi SU, Taipei (TW); Kevin Ka-Wang WANG, Gainesville, FL (US); Ronald L. HAYES, Gainesville, FL (US)

(73) Assignees: BANYAN BIOMARKERS, INC., Alachua, FL (US); THE HENRY M. JACKSON FOUNDATION FOR THE ADVANCEMEN OF MILITARY MEDICINE, INC., Bethesda, MD (US)

(21) Appl. No.: 15/441,223

(22) Filed: Feb. 23, 2017

Related U.S. Application Data
(63) Continuation of application No. 13/618,589, filed on Sep. 14, 2012, now abandoned.
(60) Provisional application No. 61/534,560, filed on Sep. 14, 2011.
(60) Provisional application No. 61/569,047, filed on Dec. 9, 2011.

(51) Int. Cl. G01N 33/68; G01N 33/543; C12Q 1/68

(52) U.S. Cl. CPC G01N 33/6893; C12Q 1/6883; G01N 33/5438; C12Q 2600/118; C12Q 2600/158; G01N 2800/301; G01N 2800/60; G01N 2800/52

(57) ABSTRACT

Life-threatening traumas such as terrorist attacks, war, disasters, mental or physical assault, severe accidents and violence frequently provoke emotional and behavioral disturbances known as post-traumatic stress disorder (PTSD) and suicide related thereto. Accurate diagnosis and treatment planning for PTSD and suicide remain difficult. The discovery of specific markers creates new opportunities for more accurate clinical assessments identifying groups that may experience better outcomes when exposed to an intervention. The present invention provides a process of detection of P-11, UBE3A, STY1, EMAP-II, SIP1, ORC5L, DCX, SCYE protein in a biological sample of a subject suspected of suffering from PTSD and/or having suicidal tendencies, and provides additional PTSD markers which are specific to gender.

### CROSS-REFERENCE TO RELATED APPLICATIONS

This application is a continuation of U.S. patent application Ser. No. 13/618,589 filed Sep. 14, 2012, which claims priority to U.S. Provisional Patent Application No. 61/534,560 filed on Sep. 14, 2011 and U.S. Provisional Patent Application No. 61/569,047 filed on Dec. 9, 2011. Each related application is herein incorporated by reference in its entirety.

### GOVERNMENT SUPPORT

This invention was made with government support under grant W81XWH-08-2-2002 awarded by United States Army Medical Research and Materiel Command. The government has certain rights in the invention.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 750582001701 SeqList.txt, date recorded: Feb. 23, 2017, size: 2,197 bytes).

### FIELD OF THE INVENTION

The present invention relates in general to the reliable detection and identification of markers produced in subjects suffering from post-traumatic stress disorder (PTSD) and provides a reliable metric for differentiating suicidal versus non-suicidal forms of the neural pathology in subjects suffering from the disorder. In particular the invention relates to processes and kits for the detection of PTSD and suicide in addition with processes of treatment and administration of therapeutics for patients suffering from the disorder. In addition, the invention provides for an in vitro diagnostic device which enables the reliable detection and identification of markers, important for the diagnosis and prognosis of PTSD and suicide.

### BACKGROUND OF THE INVENTION

Post-Traumatic Stress Disorder (PTSD) affects 7-8% of the general population of the United States and approximately 15% of veterans returning from combat. The symptoms can persist for months or decades. Unfortunately, PTSD is often misdiagnosed and left untreated in affected civilian and military individuals, disrupting the quality of their lives, their families and children, as well as our healthcare system. Even when diagnosed, the severity of PTSD progression remains difficult to treat. The cellular and molecular mechanism of this condition is still poorly understood despite extensive study of the neurobiological correlates of this disorder, along with efforts to understand the underlying pathologies.

PTSD is a severely disabling anxiety disorder which can occur after a subject has seen or experienced a traumatic event that involved the threat of injury or death and which can be found clinically in acute or chronic forms. Relevant traumatic experiences include experiencing or witnessing childhood abuse, vehicle accidents, medical complications, physical assaults, natural disasters, jail, or war. The symptoms of PTSD include intrusion of recurrent nightmares or daytime flashbacks, characterized by high anxiety; hyperarousal, meaning a constant jumpy preparation for fight or flight; and avoidance of contact with anything or anyone which might remind the patient of the trauma. Acute PTSD may resolve within 3-6 months, however, chronic PTSD is a waxing and waning disorder that can persist for months, years, or decades. PTSD is often co-morbid with other psychiatric disorders, in particular depression, substance abuse, and suicidal thoughts. PTSD can therefore cause substantial disability.

Current diagnosis of PTSD is established on the basis of clinical history and subjective mental status examination, using a clinically structured interview, symptom checklists, or patient self-reports. However, with these subjective tests it remains difficult to distinguish PTSD from other psychiatric disorders, resulting in difficult treatment decisions as to both treatment interventions and a more definitive understanding of the etiology. The existing limitations of current clinical assessment would benefit substantially from a more objective means to enhance the ability to identify the PTSD patient, and therefore be able to differentiate PTSD from other psychiatric disorders.

Suicide is among the 10 leading causes of death for all ages and it is also among the top three causes of death for people aged 15-34. Suicidal tendencies are extremely difficult to diagnose, as well as the progression of those tendencies. Usually suicidal tendencies can only be preventatively diagnosed through a mental status examination, but more commonly after an attempt at suicide was made. Suicide is a challenging mental health concern since all major mental disorders carry an increased risk of suicide. Psychiatric disorders, such as BP, MDD, SCZ or PTSD, are present in about 90% of people who commit suicide and contribute to most of the population at risk for suicide. The incidence of these mental disorders in suicide victims at the time of their death ranges from 87.3% to 98%.

A number of risk factors increase the chance of a PTSD sufferer attempting suicide, these factors include: 1) multiple hospitalizations, 2) depression, 3) presence of stressful life events before PTSD onset, 4) younger age at PTSD onset, 5) no free intervals between episodes ofr PTSD symptoms, 6) female, 7) sexual activity, 8) higher number of previous episodes of PTSD symptoms, and 9) cyclothymic temperament. These characteristics have been conventionally used in an attempt to identify those at higher risk for suicide throughout the course.

Suicide prevention has become another major issue for military organizations. In the U.S Army alone, the incidence of suicide has been increasing steadily during the present economic crisis and the Iraq and Afghanistan wars. Although much effort has been expended in an effort to understand the biological factors that can increase the risk of suicide and to identify the biological changes that occur in those with a propensity towards self-harm, at present, there are no objective biological markers to prevent suicide. A recent study of PTSD found a substantially increased risk of suicide and suicide attempts in PTSD patients. Sixty-two percent of individuals diagnosed with PTSD have suicidal ideation with traumatic events generally increasing a person's suicide risk. The likelihood for suicide attempts of individuals with PTSD is approximately 15 times higher than in individuals without it. Thus there exists a need for determining suicide in risk of patients who suffer from PTSD.

Treatments for PTSD include psychotherapy, in particular Cognitive Behavioral Therapy and adjunct pharmacotherapy, primarily with the serotonin-specific reuptake inhibitor (SSRI's). Many different pharmacological approaches have been investigated. One approach is based on the clinical hypothesis that Major Depressive Disorder (MDD) and PTSD may have much in common. For example, these entities share common risk factors, have overlapping symptoms, and frequently occur together. Consequently, antidepressants, such as the SSRI drugs fluoxitine (Prozac) and paroxatine (Paxil), are widely considered effective at treating some symptoms of PTSD. Other commonly administered SSRI antidepressants have included venlafaxine (Effexor), and sertraline (Zoloft).

However, patients with PTSD may also have other coexisting or co-morbid clinical problems. These co-morbid conditions have led to other types of drugs being added to the armamentarium. Commonly administered antipsychotics have included mirtazapine (Remeron), olanzapine (Zyprexa) and quetiapione (Seroquel). The beta blocker propranolol has also been used to try to block memory formation in PTSD patients. Prazosin, an α₁-selective adrenoceptor antagonist, has been reported to reduce trauma-related nightmares and sleep disturbances associated with PTSD. Thus co-morbidity, either causal or compensatory, can complicate the possible approaches to PTSD pharmacotherapy. These problems to suggest that more objective, biologically based criteria for PTSD may be needed.

Although there has been an effort to develop a clinical strategy to identify patients with PTSD, there is currently no biological assay for detecting such risk in patients. Therefore, diagnosis of patients with PTSD is only established on the basis of clinical history and mental status examination, often using a clinically structured interview, symptom checklist and patient self-report. The current clinical assessment would benefit from a more objective test.

Biomarkers are increasingly used to diagnose diseases promptly and accurately, and to identify individuals at high risk for certain conditions and tendencies even before clinical manifestations arise. There are presently no objective markers to validate the diagnosis or to serve as objective surrogate endpoints for therapy for PTSD. In the absence of clinically used markers for patients with PTSD, diagnosis has been dependent on subjective clinical assessments, or from asymptomatic diagnosis by a psychologist. Because PTSD can only be diagnosed through a personal interview of a patient, where the patient may be cognizant to give answers they know to be correct, the current methods leave it difficult to diagnose subjects suffering from these disorders. As a result, a majority of PTSD cases are often missed, misdiagnosed or left untreated in thousands of affected individuals. In some cases of these disorders being missed, the safety of the general public is also compromised.

Despite today's technology with marker analysis, there remains an unmet need for prognostic indicators that can aid in the objective detection of psychiatric disorders such as PTSD, MDD, SCZ and BP. Furthermore, there exists a need to provide a metric that will aid in identifying those patients suffering from a psychiatric disorder at diagnosing those patients who are at risk for suicide. In addition, there exists a need for a process that objectively diagnoses PTSD, a need to objectively monitor PTSD progression and suicide prevention a need for a process to detect PTSD prior to clinical manifestation, and a need for clinical intervention through the use of an in vitro diagnostic device to identify neurochemical markers to direct the proper course of treatment.

### SUMMARY OF THE INVENTION

A diagnostic tool is provided for the clinical evaluation of Post-traumatic stress disorder (PTSD) and/or suicide alone, or in combination with interview based assessments or other biological marker levels for other disorders such as MDD, SCZ and BP. Furthermore, through the use of markers, an objective metric to enhance PTSD and suicidal risk assessments is provided. Through repeated testing, feedback is provided as to the effectiveness of a lifestyle or therapeutic treatment regime.

The present invention further provides an in vitro diagnostic device specifically designed and calibrated to detect neuronal protein markers that are differentially present in the samples of patients suffering from psychiatric disorders such as PTSD, MDD, SCZ, BP and suicide. These devices present a sensitive, quick, and non-invasive method to aid in diagnosis of psychiatric disorders by detecting and determining the amount of markers that are indicative of psychiatric disorders. The measurement of these markers in patient samples, alone or in combination with patient interviews, provides information that a diagnostician can correlate with a probable diagnosis of the extent of a certain psychiatric disorder.

The present invention further provides a process to detect proteins, which are both gender specific and non-gender specific, for the detection of PTSD. Representative PTSD markers are, at least one, more than one, or all gender neutral proteins, peptides, variants or fragments thereof, specific to PTSD that is selected from: synaptotagmin 1, ubiquitin protein ligase E3A, polymerase (DNA directed), delta 1, catalytic subunit 125 kDa, small inducible cytokine subfamily E, member 1 (endothial monocyte-activating), non-metastatic cells 1 protein (Nm23A), protein linase C-like 1, nuclear protein, ataxia-telangiectasia locus, antigen identified by monoclonal antibody KI-87, phospholipase C, beta 1 (phosphoinositide-specific), potassium voltage-gated channel, and subfamily H (eag-related), member 6, P-11 and P2RX7. The markers of P-1 and P2RX& are also used to determine suicidal patients as well.

Representative male specific PTSD markers are at least one, more than one, or all male specific proteins, peptides, variants or fragments thereof, specific to PTSD that is selected from: Ubiquitin-conjugating enzyme E2L3, Fas (TNFRSF6)-associated via death domain, protein kinase, AMP activated, beta 1 non-catalytic subunit, kallikrein 10, mitogen-activated protein kinase 4, TAF6 RNA polymerase II, TATA box binding protein (TBP)-associated factor, 80 kDa, protein kinase C, alpha, DNA fragmentation factor, 45 kDa, alpha polypeptide, interferon-induced protein with tetratricopeptide repeats 4, striatin, calmodulin binding protein, phosphoinositide-3-kinase, catalytic alpha polypeptide, tumor necrosis factor receptor, superfamily, member 6, nuclear autoantigenic sperm protein (histone binding), ras homolog gene family, member A, NIMA (never in mitosis gene a)-related kinase 2, SWI/SNF related, matrix associated, actin dependent regulator of chromatin, subfamily a, member 2, aryl hydrocarbon receptor nuclear translocator, synaptosomal-associated protein, 91 kDa homolog (mouse), G1 to S phase transition 2, and integrin, alpha 2 (CD49B, alpha 2 subunit of VLA-2 receptor).

Representative female specific PTSD markers are at least one, more than one, or all female specific proteins, peptides, variants or fragments thereof, specific to PTSD that is selected from: survival of motor neuron protein interacting protein 1, plakophilin 2, secretory protein SECS, epidermal growth factor receptor pathway substrate 8, diacylglycerol kinase, theta 110 kDa, centrosomal protein 2, general transcription factor IIF, polypeptide 2, 30 kDa, neurogenin 3, and ADP-ribosyltransferase (NAD+; poly(ADP-ribose)polymerase).

A process is also provided to detect the levels of the P-11 (annexin II light chain) protein or mRNA, Ubiquitin protein ligase E3A (UBE3A), Synaptotagmin (STY1), endothelial monocyte-activating polypeptide (EMAP-II), survival of motor neuron protein interacting protein (SIP1), Origin recognition complex, subunit 5-like (ORCSL) and Doublecortex; lissencephaly, X-linked (doublecortin) (DCX) in whole blood, CNS tissue, serum, plasma, CSF, saliva, sweat, tears, urine, buccal sample or a combination thereof; and compare the measured levels of the protein to that of healthy normal subject not suffering from any psychiatric or neuronal disorder for which these markers are indicative of status; or alternatively, the historic levels from the individual provide a comparative. A diagnosis of PTSD, suicide or other of the above-cited disorders is when a level of the selected markers in a sample collected from a subject is outside a pre-selected normal range. The normal range for a given subject is selected based on patient specific variables that illustratively include age, sex, medication loadings, and interview based diagnosis. Further markers are described which are gender specific.

A process of detection of these proteins is performed through the conventional immunoassay technologies such as western blot or Enzyme-linked immunosorbent assay (ELISA), but preferably through the use of a sandwiched ELISA. Furthermore reagents such as antibodies to detect protein antigens or breakdown products of the protein are used.

It is appreciated that antigens to an aforementioned markers are used to detect the autoantibody response of the body. Use of either agent enables the detection of the amounts of the measured marker in a subject providing the metric used to detect the presence of PTSD and determine a patient's propensity for PTSD induced suicide.

P-11 mRNA in peripheral blood mononuclear cells (PBMCs) is also used to detect PTSD and other psychiatric disorders, such as MDD, to fully map the progress of the psychiatric disorder. Here the preferred process of detection is performed through a quantitative real-time polymerase chain reaction (Q-PCR) which measures the mRNA level of the marker in PBMCs from PTSD, MDD and suicide subjects. This process involves measuring P-11 mRNA levels in PBMCs of subjects, and comparing to the expression level of P-11 mRNA in samples from health individuals or non-psychiatric patients, as well as non-suicidal patients with PTSD or MDD.

The P-11 protein level in a patient sample is readily detected on a handheld or point of care platform in a clinical or field setting and largely in real time. It is appreciated that P-11 mRNA is measured independently, or in combination marker along with the P-11 protein. Through the use of either of these techniques, detected P-11 levels (mRNA or protein) significantly lower than normal for patients are indicative of those who suffer from a suicidal form of the PTSD. If detected levels of P-11 are higher than normal, the levels are indicative of a non-suicidal form of PTSD, BP, MDD or SCZ. An assessment as to a treatment regimen suitable for a subject is chosen based on the measured level of P-11 in a biological sample obtained from the subject. Financial remuneration can be requested in exchange for the measurement of the P-11 protein level or other inventive markers. At least one of the described markers is measured independently, or in combination with other PTSD markers described herein.

It is further appreciated that, the type of psychiatric disorder diagnosis is further refined subjectively through a complementary psychiatric evaluation, or objectively through the use of other markers, to the specific disorder in question. By way of example, the P2RX7 protein, P2RX7 mRNA levels or UBE3A may be used as for refinement of the diagnosis providing an additional indicator predictive factor in PTSD and suicide in patients further aiding in the differentiation of PTSD from other psychiatric disorders. It should be appreciated that the use of two or more of the markers identified provides a synergistic diagnostic test to accurately assist in the detection of suicide and/or PTSD in a subject.

### BRIEF DESCRIPTION OF THE DRAWINGS

FIG. 1 illustrates the receiver operating characteristic (ROC), Curve for ubiquitin protein ligase E3A (UBE3A), for which the AUC is 100%, for both male and female patients.

FIG. 2 illustrates the ROC Curve for synaptotagmin 1 (STY1), for which the AUC is 95% for males and 88.8% for females.

FIG. 3 illustrates the ROC Curve for small inducible cytokine subfamily E (SCYE1), for which the AUC for males is 98.1% and that for females, is 97.9%.

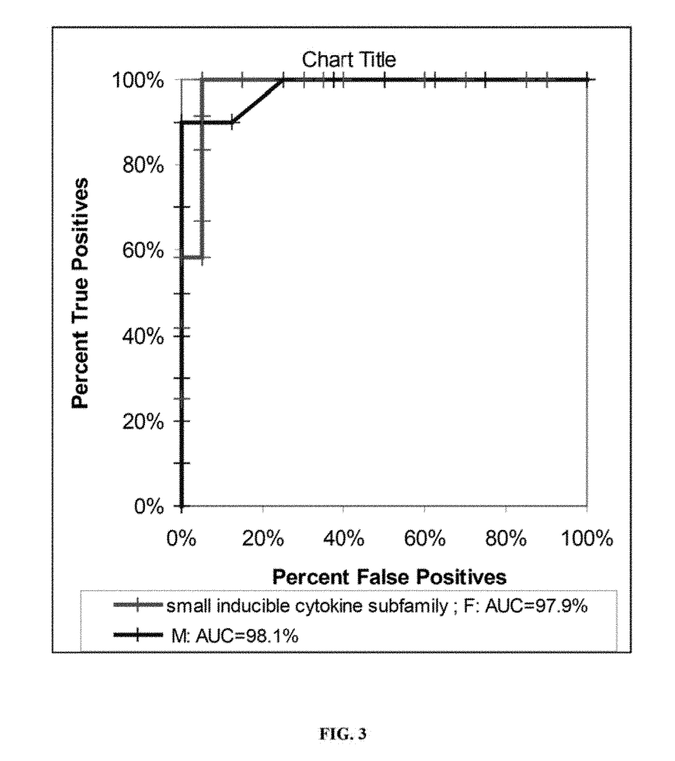

FIG. 4 illustrates the ROC Curve for the inventive markers defined between control and PTSD patients, for which the AUC is 88% for the sample demographics.

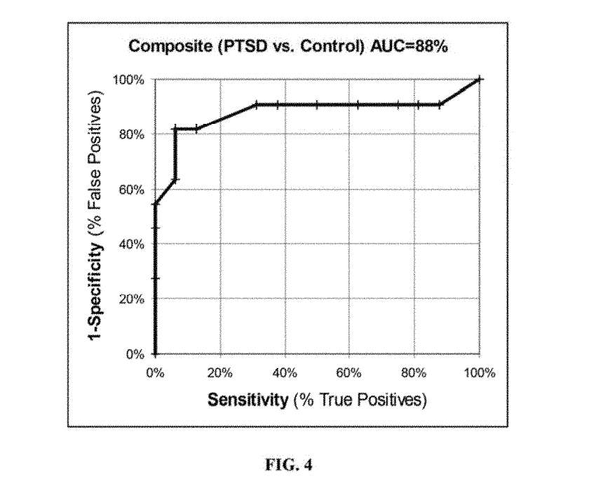

FIG. 5 shows that basal levels of blood plasma cortisol do not significantly differ between PTSD (n=13) and control subjects (n=11) (p>0.05).

FIG. 6 shows that glucocorticoid receptor (GR) mRNA levels are significantly lower in the PBMCs of PTSD than control subjects.

FIG. 7 illustrates that significantly lower P-11 mRNA levels in the patients with PTSD (n=13) compared to control (n=14). (p<0.05). PBMC P-11 mRNA levels of bipolar, BP (n=24), major depressive disorder, MDD (n=12), schizophrenia, SCZ (n=12) are significant higher than in control subjects (n=14) (p<0.001).

FIG. 8A illustrates the results from a quantitative real-time PCR analysis showing that P-11 mRNA levels significantly decreased in suicide attempters with PTSD (p<0.05) compared to control subjects. There are no significant differences in PBMC P-11 mRNA expression levels between non-suicidal patents and control subjects.

FIG. 8B illustrates P-11 mRNA levels are significantly increased in both suicide attempters and non-suicidal patients with BP (p<0.05) compared to control subjects. There are no significant differences in PBMCs P-11 mRNA expression levels between suicide attempters and non-suicidal patients with BP. ***p<0.001.

FIGS. 9A-9D shows the lack of relationships between PBMC P-11 mRNA expression levels and symptoms of suicide attempters and non-suicidal medicated patients with PTSD. FIG. 9A and B compare PBMC P-11 mRNA expression levels and Hamilton Depression Rating Scale (HAMD) or Hamilton Anxiety Rating Scale (HARS) scores in suicide attempters with PTSD. FIGS. 9C and 9D compare PBMC P-11 mRNA expression and HAMD or HARS scores in non-suicidal patients with PTSD. There are no significant correlations between HAMD or HARS scores and P-11 mRNA levels in all tested groups.

FIGS. 10A-10D shows the lack of relationships between PBMC P-11 mRNA expression levels and symptoms of suicide attempters and non-suicidal patients with BP.

FIG. 10A and 10B compare PBMC P-11 mRNA expression levels and HAMD or HARS scores in suicide attempters with BP.

FIG. 10C and 10D compare PBMC P-11 mRNA expression and HAMD or HARS scores in non-suicidal patients with BP. There are no significant correlations between HAMD or HARS scores and P-11 mRNA levels in all tested groups.

FIGS. 11A and 11B illustrate P2RX7 mRNA levels in PBMCs differ among of control subjects, suicide attempters and non-suicidal patients with PTSD or BP. FIG. 11A illustrates the results from a quantitative real-time PCR analysis which shows that P2RX7 mRNA levels are significantly decreased in both suicide attempters and non-suicide patients with PTSD (p<0.05) compared to control subjects. FIG. 11B illustrates P2RX7 mRNA levels are significantly decreased in non-suicidal patients with BP (p<0.05) compared to control subjects or suicide attempters with BP. There are no significant differences in PBMC P2RX7 mRNA expression levels found in suicide attempters with BP compared to control subjects. **p<0.01; ***p<0.001.

FIGS. 12A-12D show the relationships between PBMC P2RX7 mRNA expression levels are highly correlated with HAMD or HARS scores in non-suicidal patients with PTSD. HAMD or HARS scores are plotted against PBMC P2RX7 mRNA expression levels in suicide attempters and non-suicidal patients with PTSD. The scores are not correlated with P2RX7 mRNA levels in suicide attempters, but do correlate with P2RX7 mRNA levels in non-suicidal patients (p<0.05).

FIGS. 13A-13D show the relationships between PBMC P2RX7 mRNA expression levels are not correlated with HAMD or HARS scores in suicide attempters and non-suicidal patients with BP. HAMD or HARS scores are plotted against PBMC P2RX7 mRNA expression levels in suicide attempters and non-suicidal patients. There are no significant correlations between P2RX7 mRNA levels and HAMD or HARS scores in all tested groups.

FIG. 14 depicts the results from a quantitative real-time PCR analysis which shows that P-11 mRNA levels are significantly decreased in suicide attempters (S) and increased in suicide non-attempters (NS) compared to normal control subjects (C).

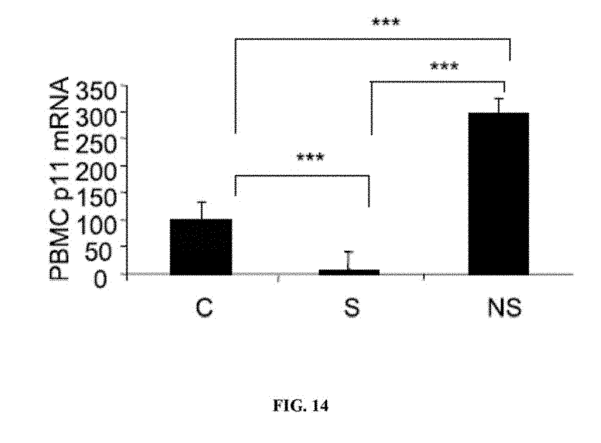

FIG. 15 illustrates the differences of P2RX7 mRNA levels in PBMCs of control subjects, suicide attempters and suicide non-attempters where quantitative real-time PCR analysis shows that P2RX7 mRNA levels are significantly decreased in both suicide attempters (S) and suicide non-attempters (NS) compared to normal controls (C).

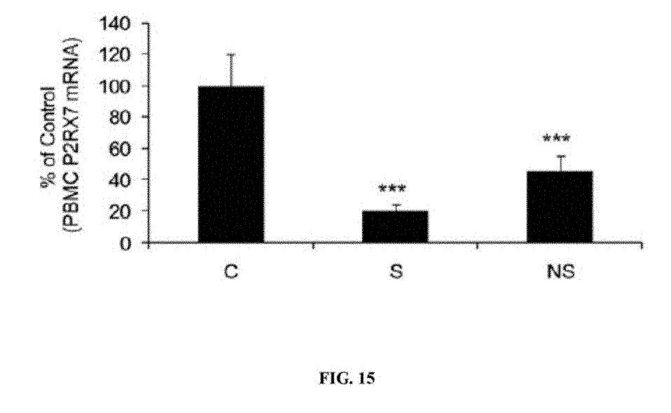

FIG. 16 is a schematic view of the in vitro diagnostic device.

### DETAILED DESCRIPTION OF THE PREFERRED EMBODIMENTS

The present invention has utility as a device and a marker test for psychiatric disorders such as PTSD and suicide thereby allowing for clinical intervention. The invention may further be used to detect neural injuries or neuronal disorders which the provided neural protein markers may be comorbid.

The following detailed description is merely exemplary in nature and is in no way intended to limit the scope of the invention, its application, or uses, which may vary. The invention is described with relation to the non-limiting definitions and terminology included herein. These definitions and terminology are not designed to function as a limitation on the scope or practice of the invention, but are presented for illustrative and descriptive purposes only. Various terms used throughout the specification and claims are defined as set forth below as it may be helpful to an understanding of the invention.

“Marker” in the context of the present invention refers to mRNA, protein or breakdown product (BDP) or an antibody to one of the aforementioned that thereof is differentially present in a sample taken from patients having neural injury and/or psychiatric disorders as compared to a comparable sample taken from control subjects (e.g., a person with a negative diagnosis, normal or healthy subject) or from a historical value of the marker for the patient.

A “breakdown product” is defined as a fragrant of an mRNA or protein that is detectable and of sufficient size to correlate to the base mRNA or protein.

The phrase “psychiatric disorder” is used herein in the broadest sense, and indicates a mental disorder that interferes with the way a person behaves, interacts with others, and functions in daily life. The Diagnostic and Statistical Manual (DSM) of Mental Disorders, published by the American Psychiatric Association, classifies psychiatric disorders such as PTSD, MDD, BP and SCZ.

The terms “patient”, “individual” or “subject” are used interchangeably herein, and is meant a mammalian subject to be treated, with human patients being preferred. In some cases, the processes of the invention find use in experimental animals, in veterinary application, and in the development of vertebrate models for disease, including, but not limited to, rodents including mice, rats, and hamsters; birds, fish reptiles, and primates.

The term “normal subject” refers to a mammalian subject, with human patients being preferred, that is not or has not suffered from neural injury manifest in psychiatric terms and does not have a history of past neural injuries or any psychiatric disorders.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers and has sufficient size to correlate with the marker. Polypeptides can be modified, e.g., by the addition of carbohydrate residues to form glycoproteins. The terms “polypeptide,” “peptide” and “protein” include glycoproteins, as well as non-glycoproteins.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

“Biological Sample” is used herein includes polynucleotides, polypeptides, peptides, antibodies fragments and correlateable breakdown products and is a bodily fluid; a soluble fraction of a cell preparation, or media in which cells are grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint; skin; or hair; and fragments of the aforementioned.

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Immunoassay” is an assay that uses an antibody to specifically bind an antigen or an antigen to bind an antibody (e.g., a marker). The immunoassay is characterized by the use of specific binding properties of a particular antibody to isolate, target, and/or quantify the antigen. It should be appreciated that many immunoassays exist and could be used interchangeably with this invention.

As used herein, the term “Traumatic Brain Injury” or “TBI” is art recognized and is intended to include the condition in which, a traumatic blow to the head causes damage to the brain, often without penetrating the skull. Usually, the initial trauma can result in expanding hematoma, subarachnoid hemorrhage, cerebral edema, raised intracranial pressure (ICP), and cerebral hypoxia, which can, in turn, lead to severe secondary events due to low cerebral blood flow (CBF). Depending upon severity, TBI may also be classified as severe, mild or moderate.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised against marker NF-200 from specific species such as rat, mouse, or human can be selected to obtain only those polyclonal antibodies that are specifically immunoreactive with marker NF-200 and not with other proteins, except for polymorphic variants and alleles of marker NF-200. This selection may be achieved by subtracting out antibodies that cross-react with marker NF-200 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

As used herein, the term “in vitro diagnostic” means any form of diagnostic test product or test service, including but not limited to a FDA approved, or cleared, In Vitro Diagnostic (IVD), Laboratory Developed Test (LDT), or Direct-to-Consumer (DTC), that may be used to assay a sample and detect or indicate the presence of, the predisposition to, or the risk of, diseases, disorders, conditions, infections and/or therapeutic responses. In one embodiment, an in vitro diagnostic may be used in a laboratory or other health professional setting. In another embodiment, an in vitro diagnostic may be used by a consumer at home. In vitro diagnostic test comprise those reagents, instruments, and systems intended for use in the in vitro diagnosis of disease or other conditions, including a determination of the state of health, in order to cure, mitigate, treat, or prevent disease or its sequelae. In one embodiment in vitro diagnostic products may be intended for use in the collection, preparation, and examination of specimens taken from the human body. In certain embodiments, in vitro diagnostic tests and products may comprise one or more laboratory tests such as one or more in vitro diagnostic tests. As used herein, the term “laboratory test” means one or more medical or laboratory procedures that involve testing samples of blood, serum, plasma, CSF, sweat, saliva or urine, buccal sample or other human tissues or substances.

A nucleic acid probe or primer able to hybridize to a target marker mRNA or is used for detecting and/or quantifying mRNA encoding a marker protein for PTSD. A nucleic acid probe can be an oligonucleotide of at least 10, 15, 30, 50 or 100 nucleotides in length and sufficient to specifically hybridize under stringent conditions to the marker protein mRNA or complementary sequence thereof. A nucleic acid primer can be an oligonucleotide of at least 10, 15 or 20 nucleotides in length and sufficient to specifically hybridize under stringent conditions to the mRNA, or complementary sequence thereof.

“Complement” and “complementary” refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

“Hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art.

“Stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and P. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

“Specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing Denhardt's solution and related chemistry such as 30% formamide incubated at 37° C. overnight followed by conventional washing.

### In Vitro Diagnostic Device

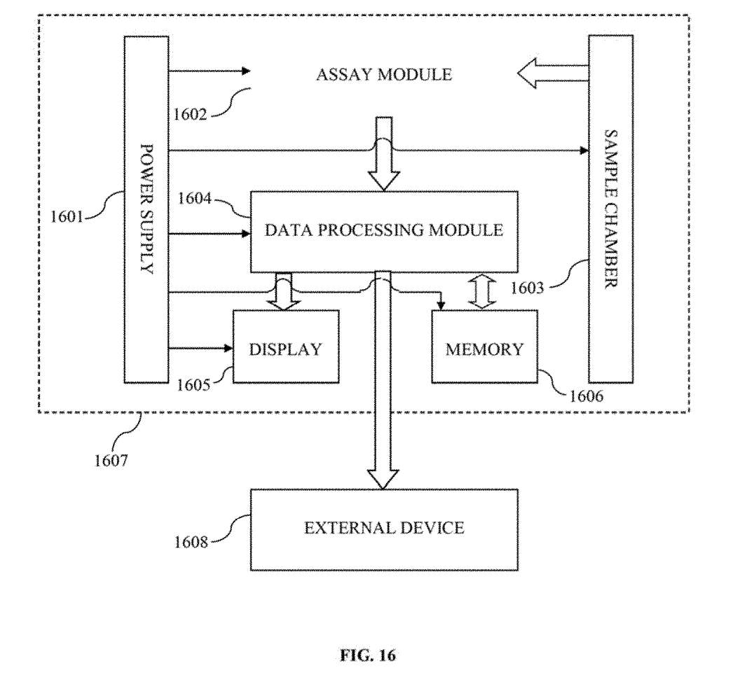

FIG. 16 schematically illustrates the inventive in vitro diagnostic device. An inventive in vitro diagnostic device comprised of at least a sample collection chamber 1603 and an assay module 1602 used to detect markers of psychiatric disorders. The in vitro diagnostic device may comprise of a handheld device, a bench top device, or a point of care device.

The sample chamber 1603 can be of any sample collection apparatus known in the art for holding a biological fluid. In one embodiment, the sample collection chamber can accommodate any one of the biological fluids herein contemplated, such as whole blood, plasma, serum, urine, sweat, saliva or buccal sample.

The assay module 1602 is preferably comprised of an assay which may be used for detecting a protein antigen in a biological sample, for instance, through the use of antibodies in an immunoassay. The assay module 1602 may be comprised of any assay currently known in the art; however the assay should be optimized for the detection of neural markers used for detecting neural injuries, neuronal disorders or psychiatric disorders in a subject. The assay module 1602 is in fluid communication with the sample collection chamber 1603. In one embodiment, the assay module 1602 is comprised of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. In one embodiment a colorimetric assay may be used which may comprise only of a sample collection chamber 1603 and an assay module 1602 of the assay. Although not specifically shown these components are preferably housed in one assembly 1607. In one embodiment the assay module 1602 contains an agent specific for detecting ubiquitin protein ligase E3A (UBE3A), synaptotagmin (STY1), endothelial monocyte-activating polypeptide (EMAP-II), survival of motor neuron protein interacting protein (SIP1), origin recognition complex, subunit 5-like (ORCSL), and boublecortex; lissencephaly, X-linked (doublecortin) (DCX), P-11, P2RX7 or any comdination fragment or breakdown product thereof. The assay module 1602 may contain additional agents to detect additional markers, as is described herein. Due to the co-morbidity of the psychiatric disorders with traumatic brain injury(TBI), the inventive IVD may also measure the same markers to correlate the presence or amount of the markers with the presence and severity of TBI.

In another preferred embodiment, the inventive in vitro diagnostic device contains a power supply 1601, an assay module 1602, a sample chamber 1603, and a data processing module 1605. The power supply 1601 is electrically connected to the assay module and the data processing module. The assay module 1602 and the data processing module 1605 are in electrical communication with each other. As described above, the assay module 1602 may be comprised of any assay currently known in the art; however the assay should be optimized for the detection of neural markers used for detecting neural injury, neuronal disorder or psychiatric disorders in a subject. The assay module 1602 is in fluid communication with the sample collection chamber 1603. The assay module 1602 is comprised of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. A biological sample is placed in the sample chamber 1603 and assayed by the assay module 1602 detecting for a marker of psychiatric disorder. The measured amount of the marker by the assay module 1602 is then electrically communicated to the data processing module 1604. The data processing 1604 module may comprise of any known data processing element known in the art, and may comprise of a chip, a central processing unit (CPU), or a software package which processes the information supplied from the assay module 1602.

In one embodiment, the data processing module 1604 is in electrical communication with a display 1605, a memory device 1606, or an external device 1608 or software package (such as laboratory and information management software (LIMS)). In one embodiment, the data processing module 1604 is used to process the data into a user defined usable format. This format comprises of the measured amount of neural markers detected in the sample, indication that a neural injury, neuronal disorder, or psychiatric disorder is present, or indication of the severity of the neural injury, neuronal disorder or psychiatric disorder. The information from the data processing module 1604 may be illustrated on the display 1605, saved in machine readable format to a memory device, or electrically communicated to an external device 1608 for additional processing or display. Although not specifically shown these components are preferably housed in one assembly 1607. In one embodiment, the data processing module 1604 may be programmed to compare the detected amount of the marker transmitted from the assay module 1602, to a comparator algorithm. The comparator algorithm may compare the measure amount to the user defined threshold which may be any limit useful by the user. In one embodiment, the user defined threshold is set to the amount of the marker measured in control subject, or a statistically significant average of a control population.

The methods and in vitro diagnostic tests described herein may indicate diagnostic information to be included in the current diagnostic evaluation in patients suspected of having neural injury, neuronal disorder or psychiatric disorder. In another embodiment, the methods and in vitro diagnostic tests described herein may be used for screening for risk of progressing from at-risk, non-specific symptoms possibly associated with psychiatric disorders, and/or fully-diagnosed psychiatric disorders. In certain embodiments, the methods and in vitro diagnostic tests described herein can be used to rule out screening of diseases and disorders that share symptoms with psychiatric disorder.

In one embodiment, an in vitro diagnostic test may comprise one or more devices, tools, and equipment configured to hold or collect a biological sample from an individual. In one embodiment of an in vitro diagnostic test, tools to collect a biological sample may include one or more of a swab, a scalpel, a syringe, a scraper, a container, and other devices and reagents designed to facilitate the collection, storage, and transport of a biological sample. In one embodiment, an in vitro diagnostic test may include reagents or solutions for collecting, stabilizing, storing, and processing a biological sample. Such reagents and solutions for nucleotide collecting, stabilizing, storing, and processing are well known by those of skill in the art and may be indicated by specific methods used by an in vitro diagnostic test as described herein. In another embodiment, an in vitro diagnostic test as disclosed herein, may comprise a micro array apparatus and reagents, a flow cell apparatus and reagents, a multiplex nucleotide sequencer and reagents, and additional hardware and software necessary to assay a genetic sample for certain genetic markers and to detect and visualize certain biological markers.

### Protein Biomarkers

The present invention provides a process to detect proteins, both gender specific and non-gender specific, for the detection of psychiatric disorders, for example PTSD and suicide. These same neural proteins may also be used to detect neural injuries and neuronal disorders, such as TBI, which is often comorbid with many psychiatric disorders. In a preferred embodiment, at least one, more than one, or all gender neutral proteins, peptides, variants or fragments thereof, specific to PTSD are detected and is selected from: synaptotagmin 1, ubiquitin protein ligase E3A, polymerase (DNA directed), delta 1, catalytic subunit 125 kDa, small inducible cytokine subfamily E, member 1 (endothial monocyte-activating), non-metastatic cells 1 protein (Nm23A), protein linase C-like 1, nuclear protein, ataxia-telangiectasia locus, antigen identified by monoclonal antibody KI-87, phospholipase C, beta 1 (phosphoinositide-specific), potassium voltage-gated channel, and subfamily H (eag-related), member 6, ubiquitin carboxyl terminal esterase L-1(UCH-L1), glial fibrilliary acidic protein (GFAP), a2-spectin breakdown products, synaptophysin, a -synuclein, neurogranin, S-1000, neurofilament proteins-F, H and N, microtubulin proteins, myelin basic proteins, and collapsin response mediated proteins(CRMPs), P-11 and P2RX7.

Male specific PTSD markers are also provided in an inventive process, where the marker is at least one, more than one, or all male specific proteins, peptides, variants or fragments thereof, specific to PTSD is selected from: Ubiquitin-conjugating enzyme E2L3, Fas (TNFRSF6)-associated via death domain, protein kinase, AMP activated, beta 1 non-catalytic subunit, kallikrein 10, mitogen-activated protein kinase 4, TAF6 RNA polymerase II, TATA box binding protein (TBP)-associated factor, 80 kDa, protein kinase C, alpha, DNA fragmentation factor, 45 kDa, alpha polypeptide, interferon-induced protein with tetratricopeptide repeats 4, striatin, calmodulin binding protein, phosphoinositide-3-kinase, catalytic alpha polypeptide, tumor necrosis factor receptor, superfamily, member 6, nuclear autoantigenic sperm protein (histone binding), ras homolog gene family, member A, NIMA (never in mitosis gene a)-related kinase 2, SWI/SNF related, matrix associated, actin dependent regulator of chromatin, subfamily a, member 2, aryl hydrocarbon receptor nuclear translocator, synaptosomal-associated protein, 91 kDa homolog (mouse), G1 to S phase transition 2, and integrin, alpha 2 (CD49B, alpha 2 subunit of VLA-2 receptor).

Female specific PTSD markers are also provided in an inventive process, where the marker is at least one, more than one, or all female specific proteins, peptides, variants or fragments thereof, specific to PTSD is selected from: survival of motor neuron protein interacting protein 1, plakophilin 2, secretory protein SEC8, epidermal growth factor receptor pathway substrate 8, diacylglycerol kinase, theta 110 kDa, centrosomal protein 2, general transcription factor IIF, polypeptide 2, 30 kDa, neurogenin 3, and ADP-ribosyltransferase (NAD+; poly(ADP-ribose)polymerase).

Still further markers for PTSD include Ubiquitin protein ligase E3A (UBE3A), Synaptotagmin (STY1), endothelial monocyte-activating polypeptide (EMAP-II), survival of motor neuron protein interacting protein (SIP1), Origin recognition complex, subunit 5-like (ORCSL), and Doublecortex; lissencephaly, X-linked (doublecortin) (DCX).

Ubiquitin protein ligase E3A (UBE3A) is a highly relevant gene for the human brain, because mutations in the brain-imprinted maternal allele lead to mental retardation and other symptoms associated with the Angelman syndrome. By contrast, duplication of the wildtype UBE3A gene is associated with autism in humans, and transgenic overexpression of human UBE3A in the mouse brain is associated with a reduction in the number and lengths of dendritic spines. Consistently, over-expression of the highly conserved Drosophila homologue for UBE3A (dUBE3A) in the mouse brain causes decreased dendritic branching. In addition, expression of the imprinted UBE3A gene is detected mainly in hippocampus, and also in cerebellar Purkinje cells and the olfactory bulb. Thus, there seems to be an optimal set-point for UBE3A expression in the brain, above or below which suboptimal function becomes apparent. The high levels of UBE3A in the PTSD CSF, blood, urine, saliva or tissue is therefore a manifestation of an aberrant central process. The dysfunction is focused on the hippocampus where UBE3A expression is customarily highest in the brain. The present invention identifies elevated levels of UBE3A in whole blood, plasma, serum, CSF, urine, saliva, and brain tissue such as hippocampal or ipsilateral cortex) in patients suffering from PTSD, as compared to normal controls or historic levels for the individual.

Synaptotagmin-1 is a 57 kDa a glycoprotein containing two C2 domains related to protein kinase C and sites for palmitoylation and binding of acidic phospholipids, calcium, and calmodulin. Synaptotagmin-1 participates in the process of vesicular trafficking and exocytosis by inducing local Ca2+-dependent buckling of the plasma membrane. Synaptotagmin-1 is a transmembrane component of synaptic vesicles, which has been implicated in regulating the process of calcium dependent membrane fusion occurring during exocytotic neurotransmission. The intact 65 kDa synaptotagmin-1 protein has been identified in human cerebrospinal fluid (Davidsson et al, 1996), where it is found to be reduced in CSF from patients with early onset Alzheimer disease (EAD). Concomitantly, it has been reported reduced levels of synaptotagminl in post-mortem hippocampus and frontal cortex of EAD patients. The present invention identifies decreased levels of SYT1 protein in whole blood, plasma, serum, CSF, urine, saliva, and brain tissues such as hippocampus and ipsilateral cortex in patients suffering from PTSD as compared to normal controls or historical levels for the individual, and represents a partially protective function occurring in the hippocampus of the PTSD brain.

Endothelial monocyte activating proteins (EMAP-II) is an inflammatory cytokine. Its pro EMAP-II precursor is identical to the auxiliary p43 component of the aminoacyl-tRNA synthetase complex. EMAP-II domain of p43 is released readily from the complex after in vitro digestion with caspase 7 and is able to induce migration of human mononuclear phagocytes. P43 compares well with a molecular fuse that triggers the irreversible cell growth/cell death transition induced under apoptotic conditions. EMAP cytokine is released from the mammalian multisynthetase complex after cleavage of its p43/pro EMAP-II component.

EMAP-II may also be known as Small Inducible cytokine subfamily E, member 1 (SCYE1) has multiple names and functions, based on its initial discovery as part of a aminoacyl-tRNA synthase complex, and then later as a protein which is induced by apoptosis, and controls angiogenesis, inflammation and wound healing. With respect to function in the central nervous system, SCYE1 is a toll-like receptor 4 (TLR4)-dependent chemoattractant for human microglia, and in this way mediates neuronal injury. Spinal cord injury in rats is associated with accumulation of SCYE1 at the injury site, where prolonged accumulation leads to the pathophysiology of secondary injury. Our sense therefore is that, to the extent that SCYE1 protein in the CSF represents proportional expression in the substance of the brain, a reduction in SCYE1 expression would appear to fall into the category of compensatory and protective. The present invention identifies decreased levels of SCYE1 or EMAP-11 protein in whole blood, plasma, serum, CSF, urine, saliva, and brain tissue (hippocampus and ipsilateral cortex) in patients suffering from PTSD as compared to normal controls.

Survival of motor neuron protein interacting protein (SIP1; Gemin 2) causes spinal muscular atrophy and is also associated with motor neuron degeneration. The biochemical function of both Survival of Motor Neuron protein (SMA) and SIP1 is to aid in pre-mRNA splicing. The present invention identifies decreased levels of SIP1 protein in whole blood, plasma, serum, CSF, urine, saliva, and brain tissue (hippocampus and ipsilateral cortex) in patients suffering from PTSD as compared to normal controls.

Origin recognition complex, subunit 5-like (ORCSL) protein function is to form a complex with 5 other proteins which initiates DNA replication. This protein has many other functions, including a role in gene silencing and heterochromatin formation. The reason why this protein has a specific function in the brain is related to its position of its gene, immediately juxtaposed to reelin (RELN) on chromosome 7q22. Mutations in RELN have genetic risk factors for autism. However, these investigators show that single nucleotide polymorphisms (SNPs) for RELN, flanked by ORC5L on one side and a third gene on the other, are inherited as a haplotype in linkage disequilibrium with autism risk. In addition, SNPs in the ORC5L gene itself are also in linkage disequilibrium with autism risk. The relationship between reelin SNPs and autism risk has been verified. Taken together with the autism connection to UBE3A, the parallel deficiency of both ORC5L and UBE3A are excellent individual and combination indicators of PTSD. The present invention identifies decreased levels of ORC5L protein in whole blood, plasma, serum, CSF, urine, saliva, and brain tissue (hippocampus and ipsilateral cortex) in patients suffering from PTSD as compared to normal controls.

Doublecortex; lissencephaly, X-linked (doublecortin) (DCX) DCX is responsible for guiding neurons in the developing cortex as they migrate over long distances to reach the site of their final differentiation. Mutations in this gene are responsible for X-linked lissencephaly. Lissencephaly, caused by mutations in DCX, is a severe human neuronal migration defect characterized by a smooth cerebral surface, mental retardation and intractable epilepsy. Possibly of relevance to sleep-related symptoms of PTSD is the finding of high levels of DCX in the adult rat suprachiamatic nucleus, where circadian clock function is localized. Following traumatic brain injury, upregulation of DCX has also been shown to correlate well with better outcome in rats and children. Thus DCX is another one of those proteins, like SIP1/Gemin 2, for which low levels of expression, is not good for CNS function. The present invention identifies decreased levels of DCX protein in whole blood, plasma, serum, CSF, urine, saliva, and brain tissue such as hippocampus and ipsilateral cortex in a patient suffering from PTSD as compared to normal controlsor historic levels in the patient.

P-11 (annexin II light chain) is a member of the S-100 calcium binding protein family. P2RX7 is a human purinergic receptor, which functions as a ligand-gated ion channel triggering ATP-dependent lysis of macrophages through formation of plasma membrane pores permeable to large molecules. ATP-induced activation of this receptor can be coupled to changes in gene expression. To date, neither P-11 nor P2RX7 proteins have been identified as novel markers for PTSD, BP or suicide. Prior to present invention, no suicide biological marker had been used at the clinical sites. The role of P-11 in PTSD and there was limited to the laboratory result that stress or the expression of the stress hormone, glucocorticoid, is up-regulated in the stressed animal brain, through a glucorticocoid binding sites in the promoter region of P-11 gene. Meanwhile, stress-induced P-11 mRNA expression is attenuated by the glucocorticoid receptor antagonist RU486, evidencing that stress-induced P-11 over expression is mediated by the glucorticoid receptor. These discoveries of the molecular mechanism of P-11 in stress have led to an understanding of the role of the P-11 protein in PTSD and to the development a diagnostic tool. The present invention identifies the levels of P-11 protein or mRNA in a biological sample as a marker for posttraumatic stress disorder (PTSD) and the diagnosis of suicide in subjects with PTSD and MDD. The P2RX7 protein or mRNA is optionally used to determine bipolar disorder (BP) and otherwise objective refine the psychiatric state of a patient. An inventive process measures P-11 and optionally P2RX7 levels using agents which specifically and independently bind to the protein, mRNA or a complementary sequence thereto.

The process of detection is optionally performed using a Western Blot analysis or an Enzyme-linked immunosorbent assay (ELISA). Preferably, when ELIA is used, a sandwich ELISA is used. The P-11 protein levels are compared to levels of protein or mRNA in normal or non-psychiatric patients as well as suicide non-attempters with PTSD and BP or historic levels for the same patient. The present invention may also be used to detect psychiatric or anxiety disorders other than PTSD or BP, such as MDD and SCZ. Levels of the P-11 marker along with other markers, such as P2RX7, are used to differentiate among these different psychiatric disorders.

mRNA is optionally used as a combination marker with the proteins. In this embodiment real-time polymerase chain reaction (PCR) measures the mRNA level of the marker in peripheral blood mononuclear cells (PBMCs) from suicide attempters with mental disorder for both P-11 and P2RX7 and compared with levels of the mRNA in samples from normal or non-psychiatric patients as well as suicide non-attempters.

### Kits

The process of diagnosing psychiatric disorders may also be included as part of a kit for use in an ELISA or Western Blot, a bench top platform, a point of care device, or handheld device for diagnosing PTSD, suicide or other psychiatric disorders. The PTSD markers can also be used to screen for therapeutic targets for treating PTSD and to monitor a patient's progression or recovery from PTSD.

In certain embodiments, the diagnostic process and kits includes one or more antibodies that bind to a protein identified as specific to a PTSD or suicide cluster. The diagnostic process and kits also comprise two or more, three or more, four or more, five or more, six or more, seven or more, eight or more, nine or more, or ten or more agents or antibodies that bind to a protein identified as specific to a PTSD cluster to diagnose PTSD in a patient.

It should be appreciated that although blood and CSF are illustrated in the following Examples the inventive markers for PTSD may be detected identically using the same procedures identified, the only difference being how the biological sample is drawn, as the varying biological samples have different methods for collection as one having skill in the art should readily know.

An inventive kit is also provided for aiding a diagnosis of a psychiatric disorder wherein the kits can be used to detect any number of the diagnostic proteins of the present invention. For example, the kits can be used to detect whether the diagnostic protein markers are present in samples of a patient and normal subjects. An inventive kit is used to identify compounds that modulate expression of one or more of the markers using in vitro or in vivo animal models to determine the effects of treatment. An inventive kit includes (a) a composition or panel of markers; (b) a protein substrate; and (c) a detection reagent. Such kits are prepared from the materials described above, and the previous discussion regarding the materials (e.g., antibodies, detection reagents, immobilized supports, etc.) is fully applicable to this section and will not be repeated. Optionally, the kit includes pre-fractionation spin columns. In some embodiments, the kit optionally further includes instructions for reacting the agent with the biological sample, or other operation parameter to afford a diagnosis of the condition. The instructions, in the form of a label or a separate insert.

An inventive diagnostic kit for use in screening serum containing antigens of the polypeptide of the invention. The diagnostic kit includes an isolated antibody specifically immunoreactive with polypeptide or polynucleotide antigens, and a defection chemistry to identify the binding of the polynucleotide or polypeptide antigen to the antibody. It is appreciated that “isolated” includes quantities of impurities or spectator species that do not preclude target binding. It is appreciated that the antibody is attached to a solid support. It is appreciated that the antibody is optionally a monoclonal antibody. The detection chemistry of the kit is optionally a second, labeled monoclonal antibody. Alternatively, or in addition, the detection chemistry optionally includes a labeled, competing antigen.

In one diagnostic configuration, test serum is reacted with a solid phase reagent having a surface-bound antigen obtained by the processes of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment processes generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates is optionally used in conjunction with biotinylated antigen(s).

A kit is also provided that includes (a) a substrate with an adsorbent thereon, wherein the adsorbent is suitable for binding a marker, (b) any marker of the present invention to be tested, and (c) instructions to detect the marker or markers by contacting a sample with the adsorbent and detecting the marker or markers retained by the adsorbent. In some embodiments, the kit includes an eluent (as an alternative or in combination with instructions) or instructions for making an eluent, wherein the combination of the adsorbent and the eluant allows detection of the markers using gas phase ion spectrometry. Such kits are prepared from the materials described above, and the previous discussion of these materials (e.g., probe substrates, adsorbents, washing solutions, etc.) is fully applicable to this section and is not repeated.

A kit is also provided that includes a first substrate with an adsorbent thereon such as a particle functionalized with an adsorbent and a second substrate onto which the first substrate is positioned to form a probe which is removable and insertable into a gas phase ion spectrometer. The kit optionally includes single substrate which is in the form of a removable and insertable probe with adsorbents on the substrate. The kit also optionally includes a prefractionation spin column (e.g., Cibacron blue agarose column, anti-HSA agarose column, size exclusion column, Q-anion exchange spin column, single stranded DNA column, lectin column, etc.).

Optionally, the kit also optionally includes instructions for suitable operational parameters in the form of a label or a separate insert. For example, the kit may have standard instructions informing a consumer how to wash the probe after a sample is contacted on the probe. In another example, the kit may have instructions for pre-fractionating a sample to reduce complexity of proteins in the sample. In another example, the kit may have instructions for automating the fractionation or other processes.

### EXAMPLES

Reference will now be made in detail to the exemplary embodiments of the invention. These embodiments are described in sufficient detail to enable those skilled in the art to practice the invention and it is to be understood that other embodiments may be utilized and that changes may be made without departing from the scope of the invention. The following description is, therefore, merely exemplary.

### Example 1

Subjects with PTSD (n=13), BP (n=23), MDD (n=12), SCZ (n=12) and control (n=14) are diagnosed by two psychiatrists using criteria from The Diagnostic and Statistical Manual of Mental Disorders fourth edition DSM-IV. A biological sample of CSF, whole blood, plasma, serum, saliva and urine are obtained from each patient.

### Patients

Fourteen medication-free outpatients with chronic civilian PTSD (34.9±10.4 years old, 10 women) and ten non-traumatized, healthy subjects (35.3±13.1 years old, 7 women) are selected. The healthy subjects are chosen to match PTSD patients as closely as possible with respect to age, sex and BMI. Prodromal PTSD Traumas are prepubertal in 5 subjects and adults in nine. Time elapsed from trauma exposure was 26±4 years in pre-pubertal trauma, and 10.1±8.8 years in adult exposure. Patients are otherwise physically healthy, with no psychotropic medication for at least three weeks prior to lumbar puncture and do not meet criteria for alcohol or substance abuse, or dependence, for at least six months prior. However, the required medication-free period for PTSD patients is extended to six weeks for patients on paroxitine. In addition, four patients (three female and one male) are included who have a history of trauma, but without a follow-on history of PTSD. The complete demographic information on PTSD patients and healthy controls are summarized in Table 1.

TABLE 1
STUDY SUBJECT DEMOGRAPHICS

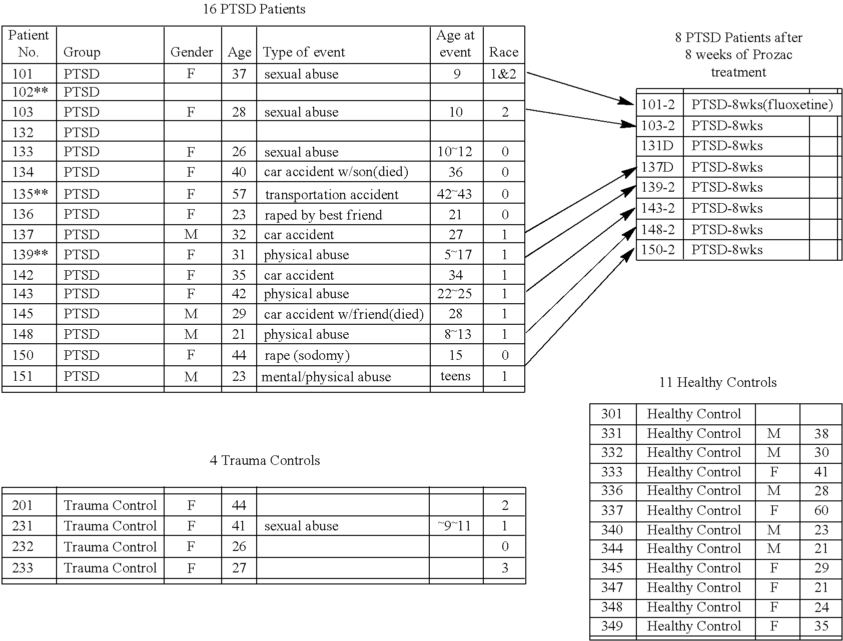

### Psychiatric Diagnoses

Psychiatric diagnoses are established using the Structured Clinical Interview for DSM-IV (SCID), and the severity of PTSD is determined using the Clinician-Administered PTSD Scale (CAPS). Severity of depressive, anxiety and overall symptoms is assessed using the Inventory of Depressive Symptomatology (IDS), Hamilton Anxiety Rating Scale (HAMA) and Clinical Global Impression-Severity scale (CGI-S), respectively. Individuals with PTSD and controls do not differ with regard to age, gender distribution, race, or body mass index (BMI). Severity of PTSD was moderate, with a CAPS score of 73.1±10.3. Depression (IDS 16.4±8.2), Anxiety (HAMA 13.1±6.8) and overall symptom severity levels (CGI-S 4±1.2) were moderate as well.

### Biological Sample Collection

Biological samples of CSF, blood, urine and saliva are collected using normal collection techniques. For CSF Lumbar Puncture (LP) was performed between 8:00 and 9:00 AM by an experienced physician. A 20-gauge introducer needle is inserted and approximately 15 cc of CSF was withdrawn and frozen in aliquots at −80° C. for later assay. For blood, the samples are drawn (10 mL each) collected by venipuncture in Vacutainer tubes, and some spun down and separated into serum and plasma. All whole blood, plasma, serum and stored in aliquots at −80° C. for later assay. For Urine and saliva, samples are collected avoiding the introduction of contaminants into the specimen is preferred. Urinalysis tubes of 8 to 15 mL are used to store samples for later use in a −80° C. freezer.

### Proteins in Biological Samples

Proteins in Biological Samples are labeled with the Cy3 fluorescent dye, and a sample of a standard samples are labeled with Cy5 to provide a common standard across all experiments. The mixture is incubated with a 507-duplicate feature antibody microarray, and imaged on a Perkin-Elmer ScanArray2 fluorescence slide reader. Biological samples are also analyzed on a Reverse Capture Protein Microarray platform. Significance is based on t-tests (p<0.05) and a local False Discovery Rate of <10%.

### Results

Based on the analysis of all PTSD patients, independent of gender, it is possible to identify those proteins which are up- or down-regulated compared to control. Table 2 lists the top 10 candidate marker proteins which fall into the statistical category of a t-test >10 −4, and a false discovery rate of <10%. Importantly, these proteins thereby satisfy both the Bonferroni correction to the P value for array data (0.05/507=10 −4) and the SAM algorithm, mentioned above. It should be noted that UBE3A, STY1, EMAP-II, SIP1, ORC5L, DCX, SCYE markers are also found to be non-gender specific markers for PTSD.

TABLE 2
PROTEINS SPECIFIC TO ALL PTSD PATIENTS

| SwissProt | ID | ttest | log Ratio | FDR |
|---|---|---|---|---|
| P21579 | synaptotagmin I | 5.9E−09 | −0.17 | 0.0% |
| Q05086 | ubiquitin protein ligase E3A (human papilloma virus E6- | 1.7E−07 | 0.48 | 0.0% |
| | associated protein, Angelman syndrome) | | | |
| P28340 | polymerase (DNAdirected), delta 1, catalytic subunit 125 kDa | 2.1E−06 | −0.13 | 0.3% |
| Q12904 | small inducible cytokine subfamily E, member 1 (endothelial | 2.2E−06 | −0.15 | 0.0% |
| | monocyte-activating) | | | |
| P15531 | non-metastatic cells 1, protein (NM23A) expressed in | 2.4E−05 | 0.26 | 2.8% |
| Q16512 | protein kinase C-like 1 | 2.8E−05 | 0.13 | 1.1% |
| Q13632 | nuclear protein, ataxia-telangiectasia locus | 4.0E−05 | 0.19 | 1.5% |
| P46013 | antigen identified by monoclonal antibody Ki-67 | 6.2E−05 | −0.20 | 0.2% |
| O9NO66 | phospholipase C, beta 1 (phosphoinositide-specific) | 6.5E−05 | 0.35 | 1.2% |
| O9H252 | potassium voltage-gated channel, subfamily H (eag-ralated), | 1.3E−04 | −0.17 | 0.0% |
| | member 6 | | | |

Using the hierarchical cluster algorithm, e proteins are separated which characterize just the male PTSD patients. The top 20 of the set of male-specific proteins that are candidate markers for PTSD are listed in Table 3. This list includes all of the proteins listed in Table 2, plus others that are uniquely elevated in CSF from just the male PTSD patients. Some of the top 10 from Table 2 are not present because they are lower in the list than these 20. However, of interest is the fact that several proteins have been pushed to a lower position by other proteins, including four that are characteristic of inflammation. These include FADD (#2); MAPKK4 (#5); PKC alpha (#7); and TNF6 (#12). With the exception of FADD, the other three are all elevated, as marked by the +value in the log ratio column. By contrast, female PTSD patients lack these proteins, but have others with non-inflammatory character. The top 9 female PTSD-specific proteins are shown in Table 4. These markers include Neurogenin 1, SMADs, SIP, MYV, TGFβ1.

TABLE 3
PROTEINS SPECIFIC TO PTSD - MALE CLUSTER

| SwissProt | ID | ttest | log Ratio | FDR |
|---|---|---|---|---|
| P51966 | ubiquitin-conjugating enzyme E2L 3 | 1.1E−11 | 0.19 | 0.1% |
| Q13158 | Fas (TNFRSF6)-associated via death domain | 7.4E−09 | −0.19 | 0.0% |
| Q9Y478 | protein kinase, AMP-activated, beta 1 non-catalytic subunit | 2.6E−08 | 0.18 | 0.0% |
| O43240 | kallikrein 10 | 5.0E−08 | 0.22 | 0.1% |
| P45985 | mitogen-activated protein kinase kinase 4 | 2.5E−06 | 0.24 | 0.0% |
| P49848 | TAF6 RNA polymerase II, TATA box binding protein (TBP)- | 2.8E−06 | 0.19 | 0.0% |
| | associated factor, 80 kDa | | | |
| P17252 | protein kinase C, alpha | 2.9E−06 | 0.20 | 0.0% |
| O00273 | DNA fragmentation factor, 45 kDa, alpha polypeptide | 1.0E−05 | 0.23 | 0.0% |
| O14879 | interferon-induced protein with tetratricopeptide repeats 4 | 1.2E−05 | 0.21 | 0.0% |
| O43815 | striatin, calmodulin binding protein | 1.8E−05 | 0.18 | 0.0% |
| P42336 | phosphoinositide-3-kinase, catalytic, alpha polypeptide | 4.2E−05 | 0.26 | 0.0% |
| P25445 | tumor necrosis factor receptor superfamily, member 6 | 1.1E−04 | 0.23 | 0.0% |
| P49321 | nuclear autoantigenic sperm protein (histone-binding) | 1.8E−04 | 0.20 | 0.0% |
| P06749 | ras homolog gene family, member A | 2.0E−04 | −0.22 | 0.0% |
| P51955 | NIMA (never in mitosis gene a)-related kinase 2 | 2.5E−04 | 0.25 | 0.0% |
| P51531 | SWI/SNF related, matrix associated, actin dependent regulator of | 2.7E−04 | 0.53 | 0.0% |
| | chromatin, subfamily a, member 2 | | | |
| P27540 | aryl hydrocarbon receptor nuclear translocator | 3.0E−04 | −0.22 | 0.0% |
| Q61548 | synaptosomal-associated protein, 91 kDa homolog (mouse) | 9.0E−04 | 0.56 | 0.0% |
| Q9NY44 | G1 to S phase transition 2 | 2.2E−03 | −0.19 | 0.0% |
| P17301 | integrin, alpha 2 (CD49B, alpha 2 subunit of VLA-2 receptor) | 2.2E−03 | −0.28 | 0.0% |

TABLE 4
PROTEINS SPECIFIC TO PTSD - FEMALE CLUSTER

| SwissProt | ID | ttest | log Ratio | FDR |
|---|---|---|---|---|
| O14893 | survival of motor neuron | 8.2E−06 | −0.36 | 0.0% |
| | protein interacting protein 1 | | | |
| Q99960 | plakophilin 2 | 8.9E−06 | −0.40 | 0.0% |
| Q96A65 | secretory protein SEC8 | 4.5E−05 | −0.15 | 0.7% |
| Q12929 | epidermal growth factor | 9.0E−05 | 0.15 | 0.0% |
| | receptor pathway substrate 8 | | | |
| P52824 | diacylglycerol kinase, | 6.1E−04 | 0.20 | 0.6% |
| | theta 110 kDa | | | |
| O60588 | centrosomal protein 2 | 6.9E−04 | −0.29 | 0.4% |
| P13984 | general transcription factor | 1.5E−03 | −0.51 | 0.0% |
| | IIF, polypeptide 2, 30 kDa | | | |
| Q92886 | neurogenin 3 | 1.9E−02 | −0.08 | 0.0% |
| P09874 | ADP-ribosyltransferase | 2.1E−02 | −0.19 | 4.0% |
| | (NAD+; poly (ADP-ribose) | | | |
| | polymerase) | | | |

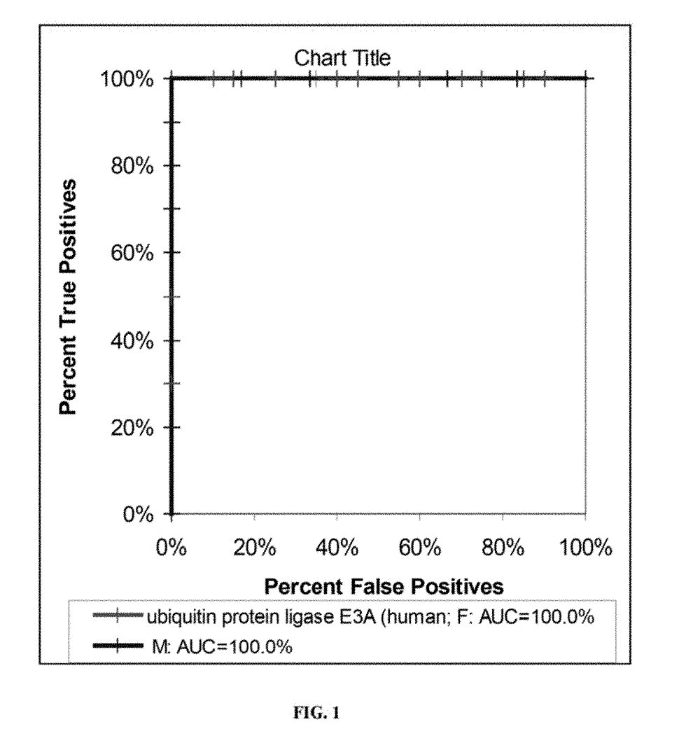

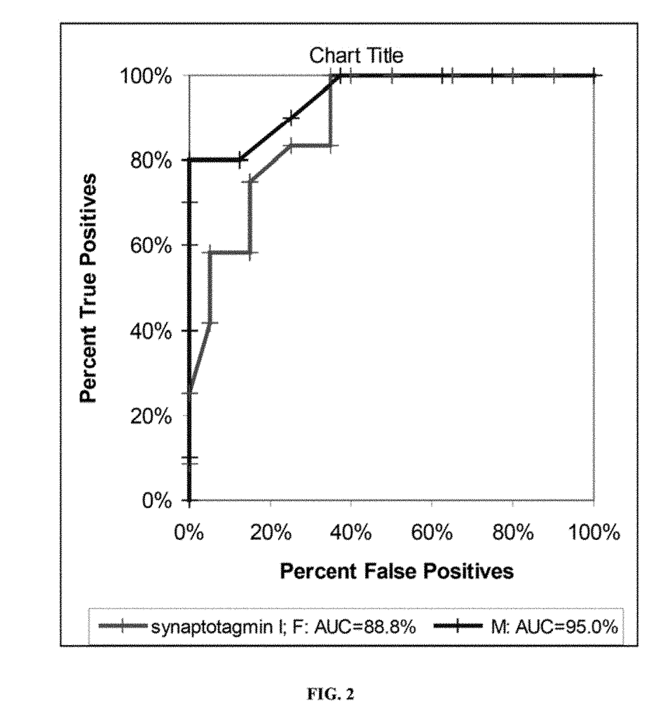

Receiver-Operating Condition (ROC) analysis is a conventional “gold standard” method of validating biological assays based on discriminating between False Positive and False Negative analytic values. The quality of an assay is based on the area under the curve (AUC), in which a value of 100% is outstanding, and a value of 50% indicates a random distribution. The top 3 candidate protein markers for gender-independent PTSD are (i) FIG. 1: ubiquitin protein ligase E3A , for which the AUC is 100%, for both male and female patients; (ii) FIG. 2: : synaptotagmin 1, for which the AUC is 95% for males and 88.8% for females; and (iii) FIG: 3: small inducible cytokine subfamily E, for which the AUC for males is 98.1% and that for females is 97.9%. FIG: 4 represents the composite ROC curves for the inventive markers defined between control and PTSD patients, for which the AUC is 88% for the sample demographics.

Several proteins are discovered which significantly distinguish between PTSD patients and Healthy Controls, independently of gender. Among the top proteins found are (i) synaptotagmin (SwissProt P21579) [p=6 EXP (−9)], a protein associated with calcium dependent neurotransmitter exocytosis. STY1 is reduced ca. 33% (p=6×10 −9) in biological samples of the PTSD patients in comparison to the healthy controls; (ii) Ubiquitin E3 Ligase (SwissProt Q05086) [p=2EXP (−7)], a protein in which mutations are associated with forms of mental retardation. UBE3A is elevated ca. 3-fold in biological samples of the PTSD patients in comparison to the healthy controls (p=2×10 −7); (iii) the Small Inducible Cytokine Subfamily E, member 1 (SCYE1/EMAPII; SwissProt Q12904) [p=2EXP (−6)]), a protein chemoattractant for microglia, and found to be elevated in spinal cord injury. SCYE1 is ca. 55% reduced in biological samples of the PTSD patients in comparison to the healthy controls (p =ca. 5×10 −13); (iv) ORCSL is ca. 30% reduced in biological samples of the PTSD patients in comparison to the healthy controls, and also has the highest significance for a difference from the healthy controls (p=5×10 −14); (v) SIP1 is reduced by ca. 52% (p =ca. 2×10 −11) in biological samples of the PTSD patients in comparison to the healthy controls; and (vi) Doublecortex (DCX) is reduced by ca. 50% (p=ca. 9×10 −6) in biological samples of the PTSD patients in comparison to the healthy controls.

In addition, we have identified different sets of proteins which significantly discriminate between Male and Female PTSD patients. Biological samples from Male PTSD patients are significantly characterized by many proteins associated with proinflammatory signaling pathways. Importantly, these proteins do not distinguish between Male and Female Healthy Controls.

These results are confirmed with quantitation of mRNA by Real-time RT-PCR About 1.5 μg total mRNA is isolated from subject blood samples and reverse transcribed in a reaction volume of 20 μl using Omniscript RT kit and random primers. The product is diluted to a volume of 150 μl and 6 μl aliquots are used as templates for amplification using conventional PCR reagent kit components and gene-specific primers. mRNA detection correlates with that of the corresponding protein for determining PTSD.

### Example 2

Blood samples are obtained from twenty six (26) psychiatric patients with post-traumatic stress disorder (PTSD) and major depressive disorder (MDD), consisting of eleven (11) patients who attempted suicide, fifteen (15) patients whom do not exhibit suicidal behaviors and fourteen (14) normal controls who do not have PTSD. The subjects are not taking anti-psychotic medication. The samples are subsequently analyzed for mRNA of P-11 and mRNA of P2RX7 in peripheral blood mononuclear cells (PBMCs) using quantitative real-time PCR. A meta-analysis of microarray data of P-11, P2RX7 and S100β from post-mortem prefrontal cortex (PFC) of patients who committed suicide (n=56) and non-suicide controls (n=61) are also tested.

PBMC P-11 mRNA levels are significantly lower in suicide attempters and higher in suicide non-attempters, when compared to normal controls. The PFC P-11 mRNA levels in suicide completers are also lower than non-suicide controls. Unlike P-11, P2RX7 mRNA levels are significantly lower than normal controls in all patients, including suicide attempters, suicide non-attempters, and suicide completers as measured in both PBMCs and PFC. In addition, S1000 expression levels in PFC did not differ between suicide completers and non-suicide controls as measured in PFC. These trends in mRNA levels detailed herein are found to correlate with other samples collected from the subjects; the other samples including cerebral spinal fluid (CSF), plasma, serum, urine, and saliva. P-11 protein and P2RX7 protein levels are also confirmed to trend as detailed above for mRNA by measurement of protein levels by ELISA on aliquots of the samples used to test mRNA levels.

TABLE 5
DEMOGRAPHIC DATA AND CONTROL LEVEL BETWEEN
PTSD AND HEALTH CONTROLS

| | | Health Control | | |
|---|---|---|---|---|
| | PTSD(N = 13) | (N = 14) | F | P |
| Age | 43.15 (SD = 12.8) | 42.2 (SD = 12.6) | | NS |
| Sex (M:F) | 1:12 | 1:10 | | NS |
| Education | | | | |
| <12 yrs | 9 (69%) | 7 (64%) | NS | |
| >12 yrs | 4 (31%) | 4 (36%) | | |
| Marital status | | | | |
| single | 4 (31%) | 4 (36%) | NS | |
| married | 6 (46%) | 7 (64%) | | |
| divorced | 3 (23%) | | | |
| Saliva cortisol | 15.6 (SD = 7.5) | 18.7 (SD = 8.3) | 0.94 | NS |
| Auc | | | | |

AD=Standard deviation; AUC=Area under curve, average by day 1 and day 2 AUC. Average concentration of plasma cortisol levels of control and PTSD are 3.86+2.33 ng/ml, and 2.96+1.88 ng/ml, respectively; Each AUC calculated 4 times (8 AM, 10AM, 4 PM, 10 PM) salivary cortisol levels.

For the mRNA markers a quantitative real time PCR analysis is used using whole blood samples. The purification of PBMC mRNA, cDNA synthesis and quantitative real time PCR are carried out according to manufacturer's protocol. Differences in the levels of P-11 mRNA, GR mRNA and cortisol are assessed by two way ANOVA (analysis of variance). Significant differences are defined as P-value of 0.05 or less. Whole blood (2.5 mL) are transferred to PAXgene tubes containing 6.9 mL of stabilization reagent and stored either at frozen at −70° C. after 2 h respite to allow hemolysis of red blood cells. The amount of total RNA is quantified using a Nano Drop spectrophotometer. The quality is electrophoretically controlled on an Agilent BioAnalyzer using the RNA 6000 Pico assay. RNA ratio values (28S/18S) are estimated and obtained from all samples. Total RNA (2.5 μg) are then reverse transcribed with random hexamers (Eurogentec) and SuperScript RT RNase H- reverse transcriptase (Life Technologies) in a final reaction volume of 45 μL. Data are shown as means+/−SEM, *p<0.05 (control vs. PTSD) and are analyzed.

Basal levels of blood plasma and saliva cortisol in PTSD patients and controls, as well as levels of P-11 and GR mRNA in PBMC of the patients with PTSD and controls are measured in the samples.

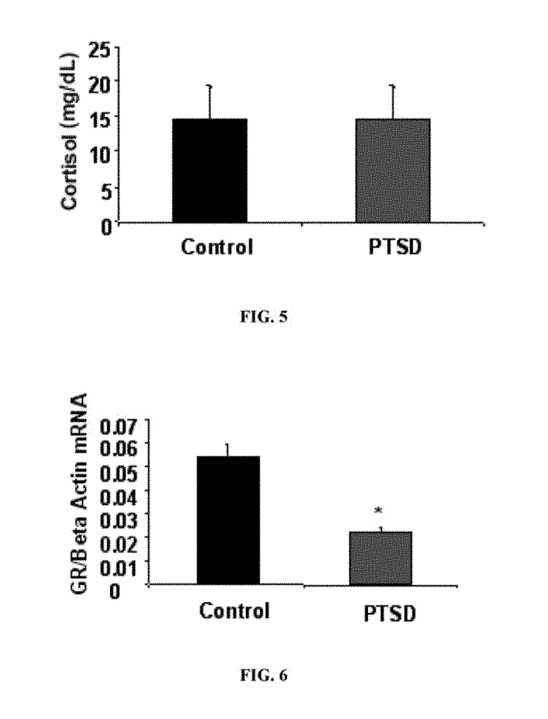

FIG. 5 shows that basal levels of blood plasma cortisol do not significantly differ between PTSD (n=13) and control subjects (n=11) (p>0.05).

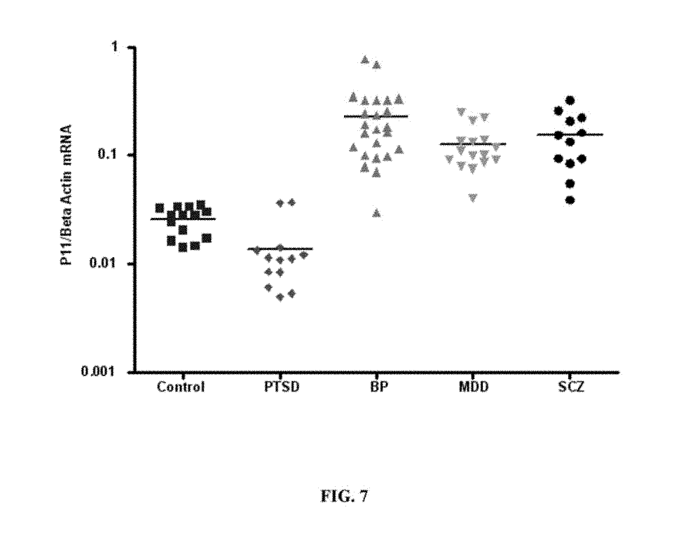

FIG. 6 shows that GR mRNA levels are significantly lower in the PBMCs of PTSD than control subjects.

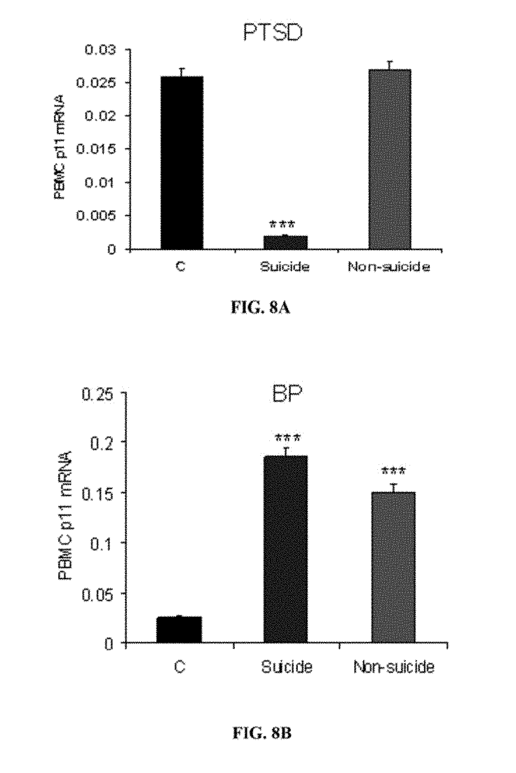

FIG. 7 shows significantly lower P-11 mRNA levels in the patients with PTSD (n=13) compared to control (n=14). (p<0.05). PBMC P-11 mRNA levels of bipolar, BP (n=24), major depressive disorder, MDD (n=12), schizophrenia, SCZ (n=12) are significant higher than in control subjects (n=14) (p<0.001).

These trends in mRNA levels detailed herein are found to correlate with other samples collected from the subjects; the other samples including cerebral spinal fluid (CSF), plasma, serum, urine, and saliva. P-11 protein and P2RX7 protein levels are also confirmed to trend as detailed above for mRNA by measurement of protein levels by ELISA on aliquots of the samples used to test mRNA levels.

### Example 3

The ability of the present invention to provide feedback as to the effectiveness of chemical and therapeutic agent interventions is provided in this example in which biological samples are obtained from a total of 49 subjects. Fourteen (14) of the subjects are determined to be suffering from PTSD, nine (9) of which are suicidal, the remaining five (5) are non-suicidal. Twenty one (21) of the subjects are determined to be suffering from BP, seven (7) of which are suicidal, the remaining fourteen (14) are non-suicidal. The remaining fourteen subjects are healthy control subjects. There are no significant group differences for gender (See Table 6). There are no statistically significant differences between BP or PTSD patients and control subjects in terms of age, gender ratio, education level or marital status. Table 6 shows that BP and PTSD patients, with and without suicide attempts, have similar average ages at onset, respectively.

All PTSD subjects are medicated with sixty six percent of suicide attempters with PTSD are taking rivotril (Clonazepam, a benzodiazepine), while 55% are taking depakine. Twenty two percent are taking either cymbalta, lendormin, modipanol, semi-nax or seroxat.

Eleven percent are taking either seroxat, abilify, akineton, ativan, effexor, lodopin, nil, olanzapine, remeron, sinequan, stilnox, tryptanol, wellbutrin or Xanax.

Sixty percent of non-suicidal patients with PTSD are taking erispan, while 40% are taking either effexor or stilnox. Twenty percent are taking either abilify, depakine, inderal, lendorm, mesyrel, nil, rivotril or seroxat. None of the BP patients are medicated.

TABLE 6
Demographic data of suicide attempters and non-suicidal patients
with BP or PTSD and control subjects.

| | Groups | | |
|---|---|---|---|
| | Suicide | Non-suicidal | |
| Patient Groups | attempters | patients | Differences |
| PTSD (age) | 38.57 ± 4.8 | 47.56 ± 3.0 | P = 0.452, df = 3.31, |
| | (20-56) | (42-69) | NS |
| BP (age) | 42.71 ± 4.3 | 50.15 ± 3.9 | |
| | (29-57) | (23-80) | |
| Sex of PTSD | 1:8 | 1:4 | P = 0.604, (df = 8.4) |
| (m/f) | | | NS |
| Sex of BP | 0:7 | 1:13 | P = 0.667, (df = 6.13), |
| (m/f) | | | NS |

NS, non-significant

Diagnoses of test subjects are established by two psychiatrists using the Mini International Neuropsychiatric Interview (MINI) and DSM-IV for all study subjects. All patients meet DSM-IV diagnostic criteria for BP or PTSD. Exclusion criteria are current medical problems, significant physical illness, neurological diseases, history of head trauma with loss of consciousness, and history of substance abuse and current alcohol abuse (within 6 months). Non-psychiatric control subjects are matched as a group, with PTSD and BP patients for age, gender, education, and race. Lifetime history of suicide attempts is assessed by using a semistructured interview and combining a review of medical records.

To quantify the clinical outcome of mood and anxiety symptoms, The Hamilton Rating Scale for Depression (HAMD), and Hamilton Anxiety Scale (HARS) are used, respectively.

Heparinized and unheparinized blood samples (10 mL each) are collected by venipuncture in Vacutainer tubes. Peripheral blood mononuclear cells (PBMCs) are separated by centrifugation on Ficoll-Hypaque (Invitrogen) density gradient. The blood samples are stored at −80° C.

The whole blood samples are centrifuged at 1300×g for 10 minutes at 4 degrees. And then the plasma is transferred to a labeled fresh Eppendorf tube and stored at −80° C. Plasma levels of P-11 are determined by highly sensitive ELISA. A monoclonal anti-human-P-11 antibody is used. P-11 concentration is determined from the regression line for the P-11 standard curve conducted under similar conditions in each assay.

RNA is extracted from human blood lysates using PAXgen blood RNA validation Kit (PreAnalytiX a Qiagen/BD company, Valencia, Calif.). cDNA is generated from 3 mg of total RNA using Superscript III RT (reverse transcriptase) and oligo (dT) primers (Invitrogen). Real-time PCR is performed on the generated cDNA product in the IQ5 sequence detection system using SYBR Green (Bio-Rad). The following sequences are used for human P-11 mRNA analyses: forward 5′AAATTCGCTGGGGATAAAGG-3′ (SEQ. ID. NO. 1) and reverse 5′AGCCCACTTTGCCATCTCTA-3′ (SEQ. ID. NO. 2) primers. The sequences for P2RX7 mRNA analyses are forward 5′AGATCGTGGAGAATGGAGTG-3′ (SEQ. ID. NO. 3), and reverse 5′-TTCTCGTGGTGTAGTTGTGG-3′ (SEQ. ID. NO. 4) primers. Beta-actin mRNA level is used as an internal control for normalizing P-11 or P2RX7 mRNA levels in control and experimental samples. The sequences for beta-actin primers are 5′-ACCTGTACGCCAACACAGTG-3′ (SEQ. ID. NO. 5) and 5′-ACACGGAGTACTTGCGCTCA-3′ (SEQ. ID. NO. 6) (Applied Biosystems). Dilution curves are used to confirm the linear dependence of the threshold cycle number on the concentration of template RNAs.

Relative quantitation of P-11 or P2RX7 mRNA in control and experimental samples are obtained using the standard curve method.

All data are presented as mean±S.D. The differences in PBMC P-11 or P2RX7 expression among suicide attempters and non-suicide patients with PTSD, BP and control subjects are analyzed by one-way ANOVA. The analysis of the correlation coefficient and P-values are also carried out. Statistics are performed using GraphPad Prism (GraphPad Software, Inc. San Diego, Calif.). SPSS (SPSS Inc. Chicago, Ill.) and Microsoft Excel.

PBMC P-11 mRNA and P2RX7 mRNA expression levels are measured for each sample and the results are compared.

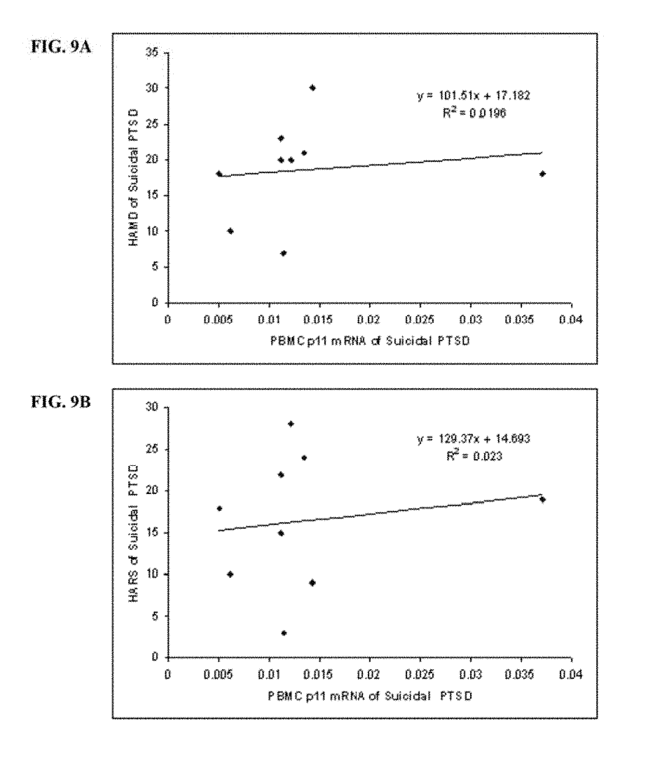

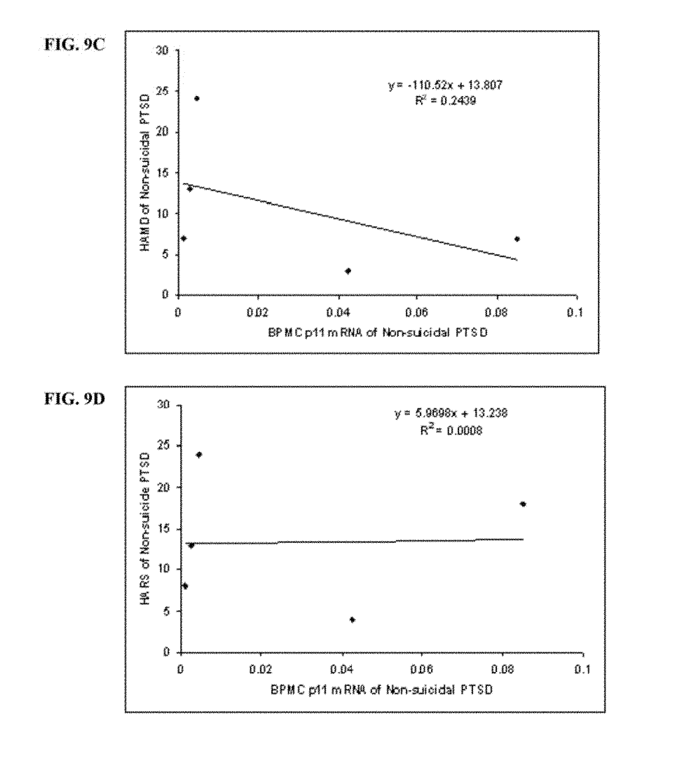

FIG. 8A demonstrates the differences in PBMC P-11 mRNA expression levels among controls, suicide attempters and non-suicidal patients with PTSD. Real-time PCR data revealed a significant difference among the groups for P-11 mRNA levels in PBMC having BP. Suicide attempters with PTSD have significantly lower levels of P-11 mRNA in PBMCs than control subjects and non-suicidal patients.

FIG. 8B demonstrates the differences in PBMC P-11 expression levels among control subjects, suicide attempters and non-suicidal patients with BP. Real-time PCR data reveal a significant difference among the groups for P-11 mRNA levels in PBMC. Both suicide attempters and non-suicidal patients exhibit significantly higher levels of P-11 mRNA in PBMCs than the control subjects, while there is no significant difference in P-11 mRNA levels between suicide attempters and non-suicidal patients.

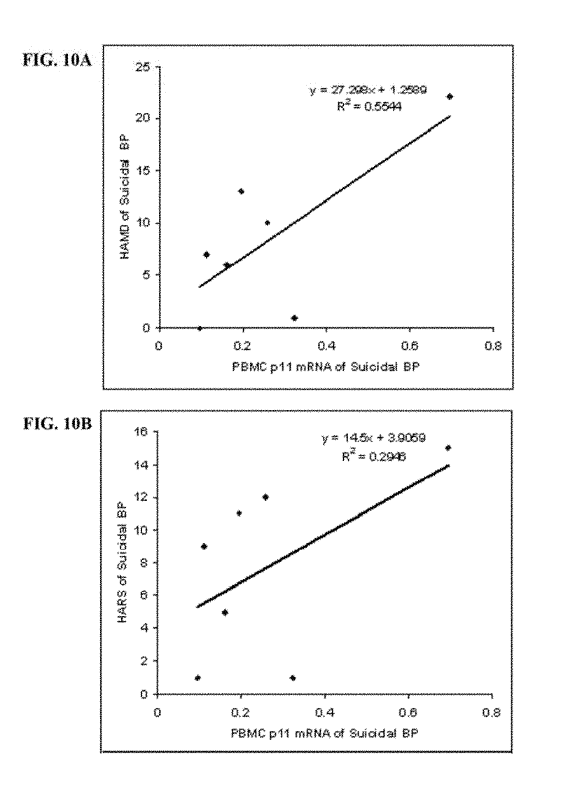

PBMC P-11 mRNA expression levels no longer correlate with symptoms of PTSD in patients with or without suicide attempts in the medicated cohort suggesting effectiveness of treatment. A correlational analysis on P-11 levels is performed in PTSD patients with their depression and anxiety as measured by the Hamilton Rating Scale for Depression (HAMD), and Hamilton Anxiety Scale (HARS). PBMC P-11 mRNA expression is not correlated with either HAMD or HARS scores in suicide attempters (FIG. 9A-B) or non-suicidal patients with PTSD (FIG. 9C-D).

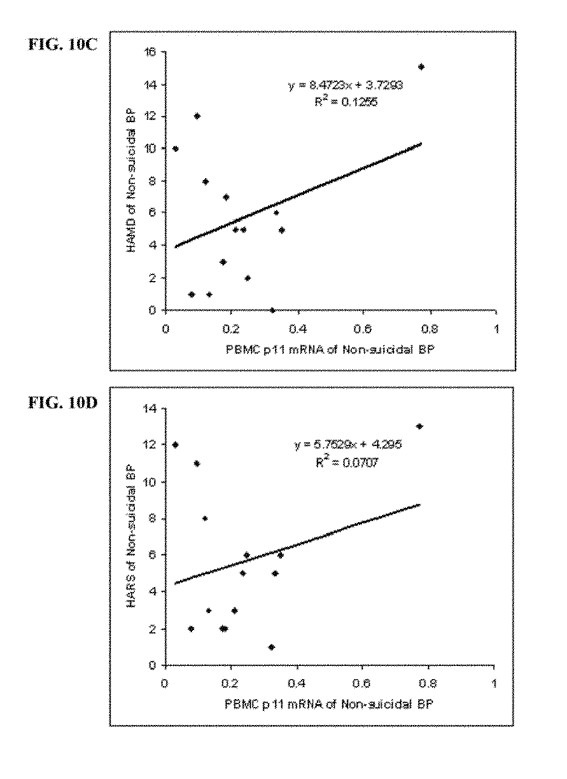

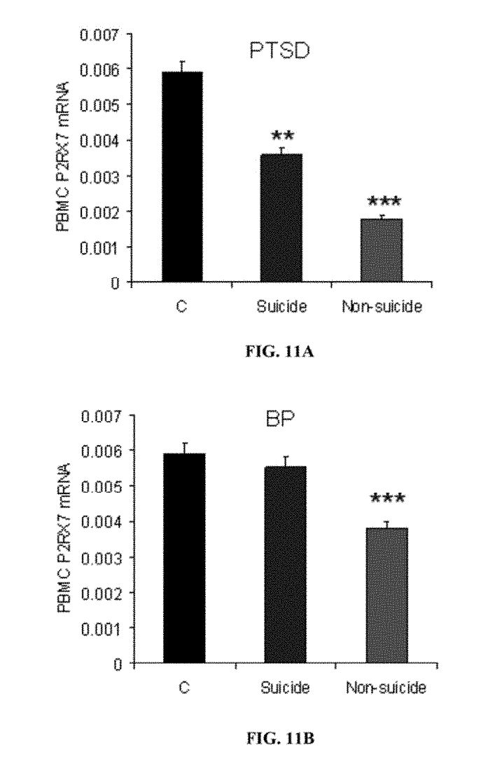

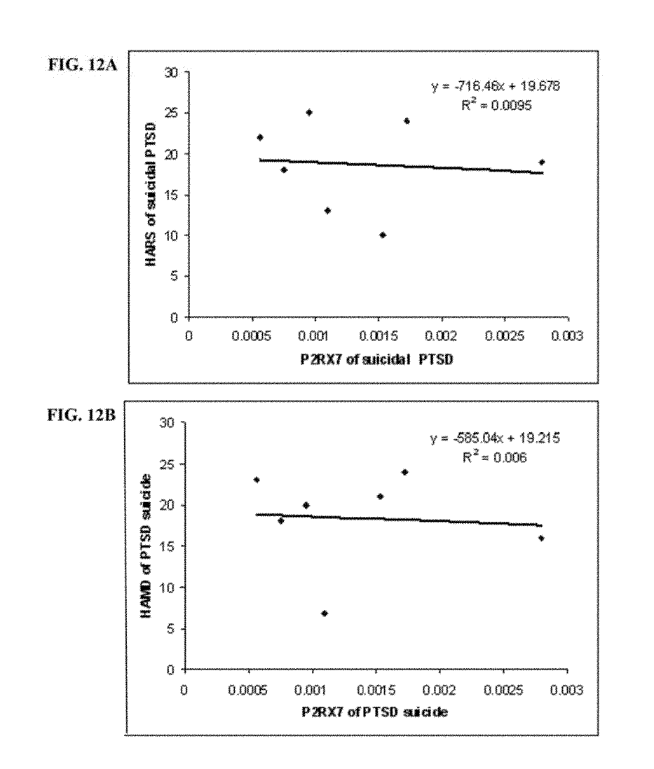

PBMC P-11 mRNA expression levels no longer correlate with symptoms of BP in patients with or without suicide attempts in the medicated cohort. A correlation analysis on P-11 levels is made in BP patients with their depression and anxiety as measured by HAMD, and HARS. PBMC P-11 mRNA expressions no longer correlated with either HAMD or HARS scores in suicide attempters (FIG. 10A-B) or non-suicidal patients with BP (FIG. 10C-D).

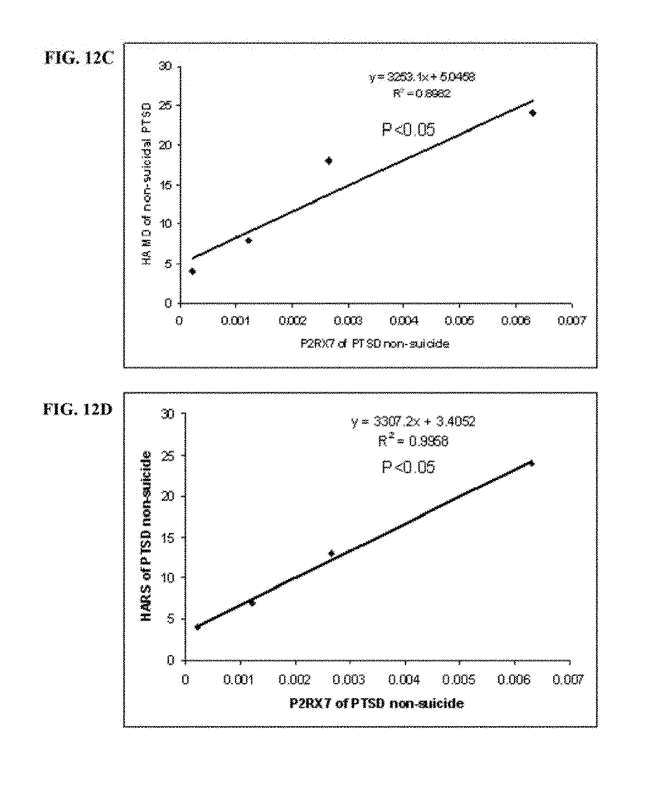

PBMC P2RX7 mRNA expression levels of PTSD patients, with or without suicide attempts, and BP patients without attempts are significantly less than those of control subjects. FIG. 11A demonstrates the differences in PBMC P2RX7 expression levels among control subjects and PTSD patients with or without suicide attempts. Real-time PCR data reveal a significant difference among the groups for P2RX7 mRNA levels in PBMC. Both suicide attempters and non-suicidal patients with PTSD had significantly lower levels of P2RX7 mRNA in PBMCs than the controls.

FIG. 11B demonstrates the differences in PBMC P2RX7 expression levels among control subjects, suicide attempters and non-suicidal patients with BP. Real-time PCR data reveal that BP patients without suicide attempts had significantly lower levels of P2RX7 mRNA in PBMCs compared with control subjects and BP patients with suicide attempts.

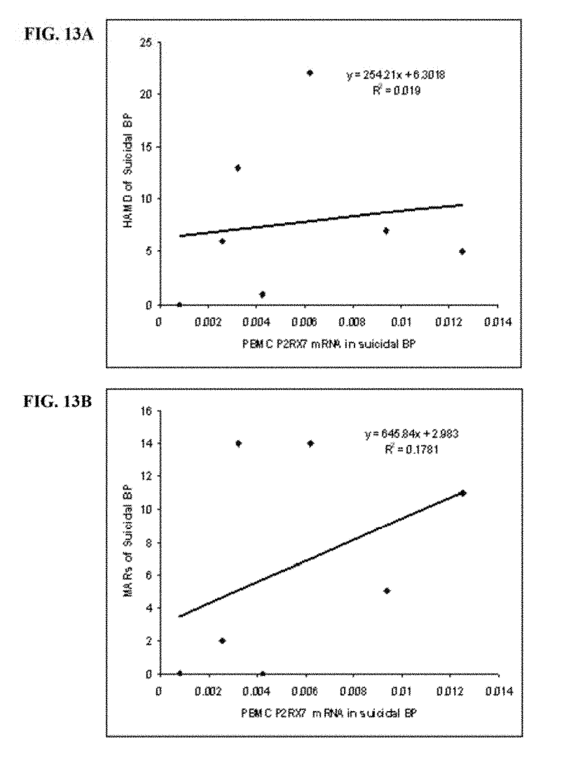

PBMC P2RX7 mRNA expression levels and symptoms of non-suicidal patients (FIG. 12C-D), but not suicide attempters (FIG. 12A-B), with PTSD are highly correlated.

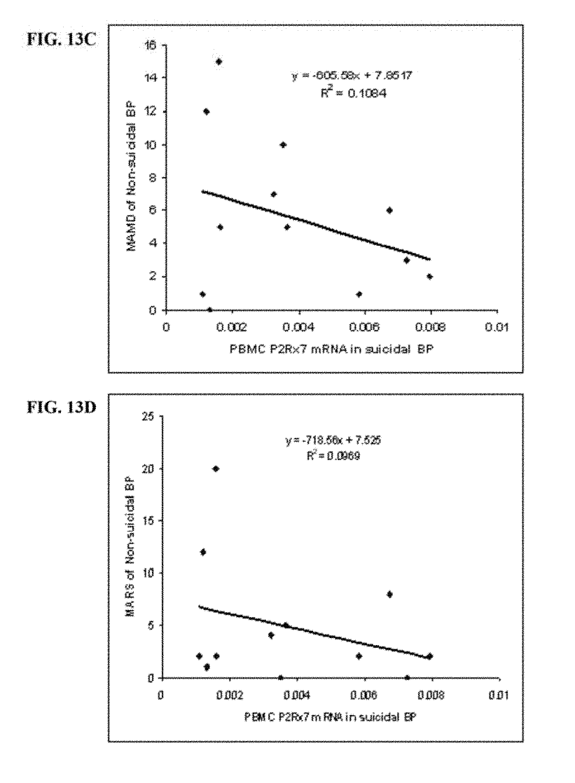

PBMC P2RX7 mRNA expression levels do not correlate with symptoms of BP patients, with or without suicide attempts. P2RX7 mRNA levels in PBMCs of BP patients with and without suicide attempts are not significantly correlated with either HAMD or HARS scores (FIG. 13A-D).

These trends in mRNA levels detailed herein are found to correlate with other samples collected from the subjects; the other samples including cerebral spinal fluid (CSF), plasma, serum, urine, and saliva. P-11 protein and P2RX7 protein levels are also confirmed to trend as detailed above for mRNA by measurement of protein levels by ELISA on aliquots of the samples used to test mRNA levels.

### Example 4

Biological samples are obtained from 40 subjects who are diagnosed with PTSD (n=14) or MDD (n=12), and 14 normal controls. Eleven (11) of the psychiatric patients had attempted to commit suicide. The available clinical records, including inpatient and outpatient, are reviewed, and all subjects are carefully interviewed regarding psychiatric history, family history of mental disorders, and history of substance abuse. Diagnoses are established by two psychiatrists using the Mini International Neuropsychiatric Interview (MINI) and DSM-IV for all study subjects. All patients met DSM-IV diagnostic criteria for PTSD or MDD. Exclusion criteria are current medical problems, significant physical illness, neurological diseases, history of head trauma with loss of consciousness, and history of substance abuse and current alcohol abuse (within 6 months). Normal controls are matched as a group with PTSD and MDD patients for age, gender, education, and race.

After the study design had been fully explained, and prior to undergoing psychiatric evaluation and blood collection, all subjects provided a written informed consent. Lifetime history of suicide attempts is assessed by using a semi-structured interview combined with a review of medical records. The clinical diagnosis for each group is shown in Table 7. It is shown that there are no statistically significant differences between the age of patients who had attempted suicide (n=11) and those who had not (n=15). Medications used by all the groups are shown in Table 8.

TABLE 7
Demographic data of suicide attempters, suicide non-attempters
and controls.

| | Suicide | Suicide | | |
|---|---|---|---|---|
| | attempters | non-attempters | Control | |
| | (N = 11) | (N = 15) | (N = 14) | P |
| Age | 41.27 ± 3.39 | 46.53 ± 3.81 | 42.2 ± 12.9 | NS |
| Sex ratio | 10:1 | 11:4 | 13:1 | NS |
| (Female:Male) | | | | |
| Marital | | | | |
| Single | 1 (10%) | 1 (7%) | 4 (29%) | <0.05 |
| Married | 5 (45%) | 9 (60%) | 10 (71%) | NS |
| Widow/divorce | 5 (45%) | 5 (33%) | 0 (0%) | NS |
| Age onset | 34.64 ± 3.46 | 31.27 ± 3.87 | NA | NS |
| Duration | 6.7 ± 1.91 | 10.3 ± 2.65 | N/A | NS |
| Diagnosis | | | | |
| PTSD | 9 (82%) | 5 (33%) | | |
| MDD | 2 (18%) | 10 (67%) | | |

TABLE 8
Medication in suicide attempters and suicide non-attempters.

| | Anti- | Mood | Anti- | |
|---|---|---|---|---|
| | depressants | stabilizers | psychotics | Anxiolytics |
| | N (%) | N (%) | N (%) | N (%) |
| Suicide attempters | 9 (45) | 7 (70) | 8 (50) | 9 (39) |
| (N = 11) | | | | |
| Suicide | 11 (55) | 3 (30) | 8 (50) | 14 (61) |
| non-attempters | | | | |
| (N = 15) | | | | |

Three post-mortem brain collections from the Stanley Medical Research Institute (SMRI), including the Neuropathology Consortium, the Array Collection, and the Depression Cohort, are used. The subjects are matched for age, gender, race, brain pH (Table 9), post-mortem interval (PMI), side of the brain and mRNA quality. The dorsolateral prefrontal cortex (PFC) is used for all microarray studies. The RNA processing protocol is that recommended by the microarray manufacturer Affymetrix. For the suicide analysis, subjects are divided into two groups including those with psychiatric disorders who committed suicide (n=56) and normal controls without psychiatric disorders (n=61). Psychiatric disorder and medication effects are adjusted in the individual study analyses using multiple regression models.

TABLE 9
Demographic data of non-suicide controls and suicide subjects.

| | Non-suicide controls | Suicide |
|---|---|---|
| Subjects | 61 | 56 |
| Bipolar disorder | 0 | 23 |
| MDD | 0 | 22 |
| Schizophrenia | 0 | 11 |
| Average age | 45 | 41 |
| Sex (male) | 42 (70%) | 31 (55%) |
| Average brain pH | 6.5 | 6.4 |

All microarray raw data are transformed using the MAS5.0 normalization algorithm. A series of quality control (QC) analyses are performed to identify microarray sample outliers before conducting the statistical analysis. Briefly, each microarray chip is subjected to Affymetrix QC metrics for chip-level parameters such as scale factor, probe perfect match/mismatch difference counts, percent present calls, control gene (GAPDH and b-actin) 50/30 ratios, and average correlation with respect to the reference distribution for those parameters across the arrays.

For the individual study analysis, each demographic and clinical variable is assessed to identify potential confounding factors using a linear model within each study. Following these demographic analyses, the suicide group is analyzed to identify a list of discriminating genes adjusted for confounding variables. The multiple regression analysis provided an adjusted fold change, standard error (SE), and p-value for each gene in each study. For the cross-study analysis, Affymetrix microarray studies (study ids: 1, 2, 3, 4, 5, 7, 14, 15 and 21) are included. The cross-study comparisons are based on scaled representations of individual study-level analyses across studies to extract the biological patterns and relationships. Consensus fold change is calculated for each gene based on a weighted combination of the individual fold changes and the SEs for the Affymetrix probesets that map to each gene across the studies. Weights are determined in a probeset-specific manner to account for the different levels of precision associated with each probeset that map to a given gene across the platforms. The weights are equal to 1 SEi, where SEi is the standard error of the ith probeset for the gene across all the studies.

To quantify the clinical expression of mood and anxiety symptoms, the Hamilton Rating Scale for Depression (HAMD), and Hamilton Anxiety Rating Scale (HARS) are used, respectively.

Heparinized and unheparinized blood samples (10 mL each) are collected by venipuncture in Vacutainer tubes. Peripheral blood mononuclear cells are separated by centrifugation on Ficoll-Hypaque (Invitrogen) density gradient. The blood samples are stored at −80° C.

Real-time PCR analysis of P-11 or P2RX7 gene expression RNA is extracted from human blood lysates using the PAXgen blood RNA validation Kit (PreAnalytiX, Qiagen/BDCompany, Valencia, Calif.). cDNA is generated from 3 mg of total RNA using Superscript III RT (reverse transcriptase) and oligo (dT) primers (Invitrogen). Real time PCR is performed on the generated cDNA product in the IQ5 sequence detection system using SYBR Green (Bio-Rad). The sequences SEQ ID NOs 1-6 are used for analysis per Example 3Dilution curves confirmed the linear dependence of the threshold cycle number on the concentration of template RNAs. The relative quantitation of P-11 or P2RX7 mRNA in control and experimental samples is obtained using the standard curve method.

All data are presented as mean S.D or S.E. The differences in PBMC P-11 or P2RX7 expression levels among patients who attempted suicide, patients who had not attempted suicide, and control subjects are analyzed by one-way ANOVA. Analyses of the correlation coefficient and P-values are also carried out using Microsoft Excel. Statistics are performed using GraphPad Prism (GraphPad Software, Inc. San Diego, Calif.).

Differences in the P-11 mRNA expression levels in the PBMCs of suicide attempters and in the PFC of suicide completers P-11 mRNA levels in PBMCs of control subjects, suicide attempters and suicide non-attempters are shown in FIG. 10A-D. P-11 mRNA levels significantly decreased in suicide attempters compared to control subjects, while in suicide non-attempters P-11 mRNA levels are significantly greater than in either control subjects or suicide attempters.

The correlations of the HAMD and HARS scores and P-11 mRNA levels of suicide attempters and non-suicide attempters are examined. There are no significant correlations between HAMD or HARS and P-11 mRNA level in medicated suicide attempters. However, there is a significant difference in HARS scores among medicated suicide attempters and suicide non-attempters. Finally, there is a significant difference in item 3 scores of HAMD between medicated suicide attempters and medicated suicide non-attempters. These results offer feedback as to efficacy of treatment as whole in modifying the marker levels relative to unmedicated PTSD subjects.

PBMC P2RX7 mRNA levels are significantly decreased in both suicide attempters and suicide non-attempters compared to control subjects FIG. 11A-B. Combined analysis of 9 Affymetrix gene expression microarray studies showed that P2RX7 mRNA levels are consistently decreased in the PFC of suicide cases as compared to non-suicide controls (adjusted p=0.03).

There is no significant correlation between HAMD or HARS scores and P2RX7 mRNA levels in suicide attempters and suicide non-attempters of PTSD and MDD.

### Example 5

Adult male Sprague-Dawley rats each weighing 150-200 g is selected. A number of rats remain undisturbed in their home cages as controls while an equal number of rats are exposed to inescapable tail shock (stress).

The stress protocol involves placing the test group of rats in a Plexiglas restraining tube (23.4 cm long and 7 cm in diameter) and exposing them to 100 inescapable electrical shocks (2.0 mA) for 5 s each, with an average intertribal interval of 60 s. The shocks are applied through electrodes taped to the tail. The number and strength of the shocks are optimized to yield a model of inescapable stress as measured by changes in behavior and by elevated plasma corticosterone levels. The number of animals used and their suffering are minimized. The total stress session lasts approximately 100 minutes. After stress or termination, all animals are returned to their home cages.

All rats are anesthetized with a brief exposure to Isoflurane immediately or 48 h after inescapable tail shock. Their brains are quickly removed after decapitation. All dissections are performed on a frosted glass plate placed on top of crushed ice. Brain samples, such as prefrontal cortex, hippocampus, amygdala and cerebellum are quickly frozen on dry ice and stored at −70° C. until time of use. Blood is collected and frozen.

Plasma corticosterone of non-stressed control or stressed groups is measured using appropriate Enzyme Immunoassay Kits, such as the DSL-10-81100 ACTIVE Rat Corticosterone (Diagnostic Systems Laboratories, Inc., Webster, Tex., USA).

Plasma P-11 protein is measured using Goat anti-mouse IgG Microplate (R&D Systems). In order to prepare the plate, first, 100 ul of 1:1000 diluted mouse anti-human S100A10 (P-11) monoclonal antibody (Abcam, Ab52272) is transferred into each well of the ELISA plate. The plate is sealed with film and incubated overnight at room temperature. Secondly, 100 ul of the dilutions of unknowns and standards are added in the plate and incubated 2 hours at room temperature. Thirdly, 100 ul of rabbit anti-human P-11 antibody (diluted with 1:1000) (Proteintech Group, Cat. #: 11250-1-AP) is added in the plate and incubated overnight at 4° C. Fourthly, 100 ul Streptavidin-HRP (anti-rabbit IgG, R&D Systems, Cat. #: DY998) is incubated for 20 minutes at room temperature. Between the above steps, the plate is washed three times. Fifthly, 100 ul Substrate Solution (R&D Systems, Cat. #: DY999) is added in each well and incubated for 20 minutes at room temperature. Sixthly, 50 ul of Stop Solution is added and the optical density (OD) of each well is determined within 30 minutes by the microplate reader.

RNA is extracted from tissue or blood cell lysates using TRIzol. cDNA is generated from 5 ug of total RNA for each sample using Superscript III RT (reverse transcriptase) and oligo (dT) primers to exclude the possibility that differences in RNA-content could also result from differences in sample weights. Real-time PCR is performed on the generated cDNA product. The following sequences are used for human P-11 mRNA analyses: SEQ ID NOs. 1 and 2 primers. The sequences used for rat P-11 mRNA analyses are: forward 5′-TGCTCATGGAAAG GGAGTTC-3′ (SEQ ID NO. 7) and reverse 5′-CCCCGCCACTAGTGATAGAA-3′ (SEQ ID NO. 8) primers. Beta-actin mRNA level is used as an internal control for normalizing P-11 mRNA levels in control and experimental samples with SEQ ID NOs. 5 and 6 per Example 3. Dilution curves confirm the linear dependence of the threshold cycle number on the concentration of template RNAs. Measurements of P-11 mRNA in control and experimental samples are obtained using the standard curve method.

These trends in mRNA levels detailed herein are found to correlate with other samples collected from the subjects; the other samples including whole blood, cerebral spinal fluid (CSF), plasma, serum, urine, and saliva. P-11 protein and P2RX7 protein levels are also confirmed to trend as detailed above for mRNA by measurement of protein levels by ELISA on aliquots of the samples used to test mRNA levels.

Rat data for PTSD and control groups correlates with that for humans in Example 1 confirming the protocol as an animal model for human non-suicidal PTSD.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

Patent documents and publications mentioned in the specification are indicative of the levels of those skilled in the art to which the invention pertains. These documents and publications are incorporated herein by reference to the same extent as if each individual document or publication is specifically and individually incorporated herein by reference.

1. A process of determining the presence of a psychiatric disorder, comprising:
collecting a biological sample from an affected subject suspected of having a psychiatric disorder;
measuring said sample for an amount of at least one marker that is associated with a psychiatric disorder wherein the at least one marker protein is (i) synaptotagmin 1(STY1); (ii) ubiquitin protein ligase E3A; polymerase (UBE3A), (iii) delta 1, catalytic subunit 125 kDa; (iv) small inducible cytokine subfamily E, member 1 (SCYE1); (v) non-metastatic cells 1, protein (NM23A); (vi) protein kinase C-like 1; nuclear protein, ataxia-telangiectasia locus; (vii) antigen identified by monoclonal antibody Ki-67; (viii) phospholipase C, beta 1 (phosphoinositide-specific); (ix) potassium voltage-gated channel, subfamily H (eag-related), member 6; (x) endothelial monocyte-activating polypeptide (EMAP-II); (xi) survival of motor neuron protein interacting protein (SIP1); (xii) Origin recognition complex, subunit 5-like (ORCSL); (xiii) Doublecortex; lissencephaly, X-linked (doublecortin) (DCX); (xiv) ubiquitin carboxyl terminal esterase L-1(UCH-L1), (xv) glial fibrillary acidic protein (GFAP), (xvi) α2-spectrin, (xvii) synaptophysin, (xviii) α-synuclein, (xix) neurogranin, (xx) S-1000, (xxi) neurofilament proteins—F, H and N, (xxii) microtubulin proteins, (xxiii) myelin basic proteins, (xxiv) collapsin response mediated proteins (CRMPs), and (xxv) P-11, and (xxvi) P2RX7, or combinations thereof, and
comparing said amount of the at least one marker with a normal subject amount of the at least one marker measured in a normal subject not having a psychiatric disorder or a historical affected subject amount of the at least one marker;
where differential between said amount and said normal amount or said historical amount is indicative of a psychiatric disorder in said affected subject.

2. The process of claim 1 wherein the at least one marker is a protein of UBE3A, STY1, EMAP-II, SIP1, ORC5L, DCX, SCYE, UCH-L1, P-1 for combinations thereof.

3. The process of claim 1, further comprising:
selecting a male patient suspected of having post-traumatic stress disorder (PTSD), measuring said sample for an amount of at least one PTSD marker associated with PTSD, in a biological sample from a subject suspected of having a PTSD wherein the at least one PTSD protein is ubiquitin-conjugating enzyme E2L 3; Fas (TNFRSF6)-associated via death domain;
protein kinase, AMP-activated, beta 1 non-catalytic subunit; kallikrein 10; mitogen-activated protein kinase kinase 4; TAF6 RNA polymerase II, TATA box-binding protein (TBP)-associated factor, 80 kDa; protein kinase C, alpha; DNA fragmentation factor, 45 kDa, alpha polypeptide; interferon-induced protein with tetratricopeptide repeats 4; striatin, calmodulin binding protein; phosphoinositide-3-kinase, catalytic, alpha polypeptide; tumor necrosis factor receptor superfamily, member 6; nuclear autoantigenic sperm protein (histone-binding); ras homolog gene family, member A; NIMA (never in mitosis gene a)-related kinase 2; SWI/SNF related, matrix associated, actin dependent regulator of chromatin, subfamily a, member 2; aryl hydrocarbon receptor nuclear translocator; synaptosomal-associated protein, 91 kDa homolog (mouse); G1 to S phase transition 2; or integrin, alpha 2 (CD49B, alpha 2 subunit of VLA-2 receptor), and
comparing said amount of the at least one PTSD protein with a normal subject amount of the at least one PTSD marker measured in a normal subject not having PTSD or other psychiatric disorder or a historical affected subject amount of the at least one PTSD marker;
where differential between said amount and said normal amount or said historical amount is indicative of PTSD in said affected subject.

4. The process of claim 1, further comprising:
selecting a female patient suspected of having post-traumatic stress disorder (PTSD),
measuring said sample for an amount of at least one PTSD marker associated with PTSD in a biological sample from a subject suspected of having a PTSD wherein the at least one PTSD marker is survival of motor neuron protein interacting protein 1(SIP1); plakophilin 2; secretory protein SEC8; epidermal growth factor receptor pathway substrate 8; diacylglycerol kinase, theta 110 kDa; centrosomal protein 2; general transcription factor IIF, polypeptide 2, 30 kDa; neurogenin 3; or ADP-ribosyltransferase, and
comparing said amount of the at least one PTSD marker with a normal subject amount of the at least one PTSD marker measured in a normal subject not having PTSD or other psychiatric disorder or a historical affected subject amount of the at least one PTSD marker;
where differential between said amount and said normal amount or said historical amount is indicative of PTSD in said affected subject.

5. The process of claim 1, further comprising:
selecting a patient suspected of being suicidal,
measuring said sample for an amount of at least one suicide marker associated with being suicidal in a biological sample from a subject suspected of being suicidal wherein said protein marker is P-11 or P2RX7; and
comparing said amount of the at least one suicide marker with a normal subject amount of the at least one suicide marker measured in a normal subject not being suicidal or suffering from some other psychiatric disorder or a historical affected subject amount of the at least one suicide marker;
where differential between said amount and said normal amount or said historical amount is indicative of suicide in said affected subject.

6. The process of claim 1 wherein the biological sample is whole blood, plasma, serum, CSF, urine, saliva, sweat, prefrontal cortex tissue, hippocampus tissue, or ipsilateral cortex tissue.

7. The process of claim 1 further comprising: collecting successive biological samples as a function of time and monitoring for a temporal change in said amount of the at least one marker, PTSD marker or suicide marker in the biological sample of said affected subject until said amount substantially equals said normal subject amount or said historical amount of said marker as being indicative of recovery.

8. The process of claim 1 further comprising: administering a therapeutic to treat a psychiatric disorder; and collecting successive biological samples as a function of time and monitoring for a temporal change in said amount of the at least one marker, PTSD marker or suicide marker in the biological sample of said affected subject until said amount substantially equals said normal subject amount or said historical amount of the at least one marker, PTSD marker or suicide marker as being indicative of recovery and the effectiveness of the therapeutic.

9. A kit using the process of claim 1, the kit comprising:
a substrate for holding a biological sample isolated from a human subject;
an agent that specifically interacts with the at least one marker, PTSD marker or suicide marker associated with a psychiatric disorder;
printed instructions for reacting the agent with the sample or a portion of the sample to detect the presence or amount of the marker for diagnosing a psychiatric disorder in a subject.

10. The kit of claim 9, wherein the agent is an antigen which specifically and independently binds to its respective autoantibody to the at least one marker, PTSD marker or suicide marker.

11. The kit of claim 9, wherein the agent is an antibody which specifically and independently binds to its respective protein of the at least one marker, PTSD marker or suicide marker.

12. The kit of claims 9 wherein an enzyme-linked immunosorbent assay (ELISA) is used to determine whether the one or more agents bind to the at least one marker, PTSD marker or suicide marker in the biological sample.

13. An in vitro diagnostic device for detecting a psychiatric disorder in a subject, the device comprising:
a sample chamber for holding a first biological sample collected from the subject;
an assay module in fluid communication with said sample chamber, said assay module using the process of claim 1;
a power supply; and
a data processing module in operable communication with said power supply and said assay module; said assay module analyzes the first biological sample to detect at least one of said protein markers associated with a psychiatric disorder present in the biological sample and electronically communicates a presence of the marker detected in the first biological sample to said data processing module;
wherein said data processing module has an output the relates to detecting the neural injury or neuronal disorder in the subject, the output being the amount of the marker measured, the presence or absence of a psychiatric disorder, or the severity of the psychiatric disorder.

14. The device of claim 13, further comprising analyzing a second biological sample obtained from the subject, at some time after the first sample is collected, wherein if the device detects a differential amount of the measured marker in the second sample relative to the first sample an output noting the temporal change is provided by the data processing module.

15. The device of claim 13 further comprising a display in electrical communication with data processing module and displaying the output as at least one of an amount of the psychiatric marker, a comparison between the amount of the psychiatric marker and a control, presence of a psychiatric disorder, or severity of the psychiatric disorder.

16. The device of claim 13 further comprising a transmitter for communicating the output to a remote location.

17. The device of claim 1 wherein the output is digital.


SEQUENCE LISTING (8 sequences)

SEQ ID NO: 1 - DNA, 20 bp, Artificial Sequence
  forward primer for human P-11 mRNA
aaattcgctggggataaagg

SEQ ID NO: 2 - DNA, 20 bp, Artificial
  human P-11 mRNA reverse primer
agcccactttgccatctcta

SEQ ID NO: 3 - DNA, 20 bp, Artificial
  P2RX7 mRNA forward primer
agatcgtggagaatggagtg

SEQ ID NO: 4 - DNA, 20 bp, Artificial
  P2RX7 mRNA reverse primer
ttctcgtggtgtagttgtgg

SEQ ID NO: 5 - DNA, 20 bp, Artificial
  beta-actiin forward primer
acctgtacgccaacacagtg

SEQ ID NO: 6 - DNA, 20 bp, Artificial
  beta actin reverse primer
acacggagtacttgcgctca

SEQ ID NO: 7 - DNA, 20 bp, Artificial
  forward primer for rat P-11 mRNA
tgctcatggaaagggagttc

SEQ ID NO: 8 - DNA, 20 bp, Artificial
  primer
ccccgccactagtgatagaa